(19) United States
(12) Patent Application Publication
(10) Pub. No.: US 2014/0334708 A1
(43) Pub. Date: Nov. 13, 2014

(54) IMAGE PROCESSING APPARATUS AND X-RAY CT APPARATUS

(71) Applicants: Kabushiki Kaisha Toshiba, Minato-ku (JP); Toshiba Medical Systems Corporation, Otawara-shi (JP)

(72) Inventors: Yukinobu Sakata, Kawasaki-shi (JP); Kazumasa Arakita, Nasushiobara-shi (JP); Tomoyuki Takeguchi, Kawasaki-shi (JP); Nobuyuki Matsumoto, Inagi-shi (JP)

(73) Assignees: Kabushiki Kaisha Toshiba, Minato-ku (JP); Toshiba Medical Systems Corporation, Otawara-shi (JP)

(21) Appl. No.: 14/446,364

(22) Filed: Jul. 30, 2014

Related U.S. Application Data
(63) Continuation of application No. PCT/JP2013/076748, filed on Oct. 1, 2013.

(30) Foreign Application Priority Data
Oct. 1, 2012 (JP) 2012-219806

(51) Int. Cl. G06T 7/00; A61B 6/03; A61B 6/00

(52) U.S. Cl. CPC G06T 7/0012; A61B 6/5288; A61B 6/032
USPC 382/131

(57) ABSTRACT

An image processing apparatus according to an embodiment includes a generator, a selector, a first detector, and a second detector. The generator generates a group of frames corresponding to reconstructed images that correspond to a plurality of heartbeat phases of a heart. The selector specifies a corresponding frame that corresponds to a specific heartbeat phase from among the group of frames. The a first detector detects a boundary of the heart in the corresponding frame. The second detector detects a boundary of the heart in the frames other than the corresponding frame, by using the detected boundary in the corresponding frame.

### CROSS-REFERENCE TO RELATED APPLICATIONS

This application is a continuation of PCT international application Ser. No. PCT/JP2013/076748 filed on Oct. 1, 2013 which designates the United States, incorporated herein by reference, and which claims the benefit of priority from Japanese Patent Application No. 2012-219806, filed on Oct. 1, 2012, the entire contents of which are incorporated herein by reference.

### FIELD

Embodiments described herein relate generally to an apparatus which detects a boundary of a heart in acquired image data.

### BACKGROUND

Conventionally, techniques for detecting a boundary of the heart from each member of a group of frames depicting the heart have been known. For example, a boundary of the heart is detected from one frame, and subsequently, a boundary of the heart is detected from each of the rest of the frames by using the detection result. In that situation, if the accuracy of the detection from the first frame is low, there is a possibility that the accuracy levels of the detections from all the frames may become low.

### BRIEF DESCRIPTION OF THE DRAWINGS

FIG. 1 is a diagram illustrating an X-ray CT apparatus according to a first embodiment;

FIG. 2 is a flowchart according to the first embodiment;

FIG. 3 is a drawing for explaining generation of a group of frames according to the first embodiment;

FIG. 4A is a drawing of frames compliant with Digital Imaging and Communications in Medicine (DICOM) specifications according to the first embodiment;

FIG. 4B is another drawing of frames compliant with the DICOM specifications according to the first embodiment;

FIG. 5A is a drawing for explaining a boundary detecting process according to the first embodiment;

FIG. 5B is a drawing for explaining another boundary detecting process according to the first embodiment;

FIG. 6 is a diagram illustrating a system controlling unit according to a second embodiment;

FIG. 7 is a drawing for explaining a reference frame specifying process according to the second embodiment;

FIG. 8A is a drawing for explaining an X-ray detector according to the second embodiment;

FIG. 8B is another drawing for explaining the X-ray detector according to the second embodiment;

FIG. 9 is a drawing for explaining the reference frame specifying process according to the second embodiment;

FIG. 10 is a flowchart of a processing procedure in the reference frame specifying process according to the second embodiment;

FIG. 11 is a drawing for explaining another reference frame specifying process according to the second embodiment;

FIG. 12 is a diagram illustrating an image reconstructing unit according to a third embodiment;

FIG. 13 is a diagram illustrating a system controlling unit according to a fourth embodiment;

FIG. 14 is a flowchart of a processing procedure in a boundary correcting process according to the fourth embodiment;

FIG. 15 is a drawing for explaining the boundary correcting process according to the fourth embodiment;

FIG. 16 is another drawing for explaining the boundary correcting process according to the fourth embodiment;

FIG. 17 is a diagram illustrating a system controlling unit according to a fifth embodiment;

FIG. 18 is a flowchart of a processing procedure in an analysis target specifying process according to the fifth embodiment;

FIG. 19 is a drawing for explaining the analysis target specifying process according to the fifth embodiment;

FIG. 20 is another drawing for explaining the analysis target specifying process according to the fifth embodiment;

FIG. 21 is a drawing for explaining raw data in another exemplary embodiment;

FIG. 22 is a diagram illustrating an image processing apparatus according to yet another exemplary embodiment; and

FIG. 23 is a diagram of a hardware configuration of an image processing apparatus according to any of the exemplary embodiments.

### DETAILED DESCRIPTION

An image processing apparatus according to an embodiment includes a generator, a selector, a first detector, and a second detector. The generator generates a group of frames corresponding to reconstructed images that correspond to a plurality of heartbeat phases of a heart. The selector specifies a corresponding frame that corresponds to a specific heartbeat phase from among the group of frames. The a first detector detects a boundary of the heart in the corresponding frame. The second detector detects a boundary of the heart in the frames other than the corresponding frame, by using the detected boundary in the corresponding frame.

Exemplary embodiments of an image processing apparatus and an X-ray CT apparatus will be explained below, with reference to the accompanying drawings. Possible embodiments are not limited to the exemplary embodiments described below.

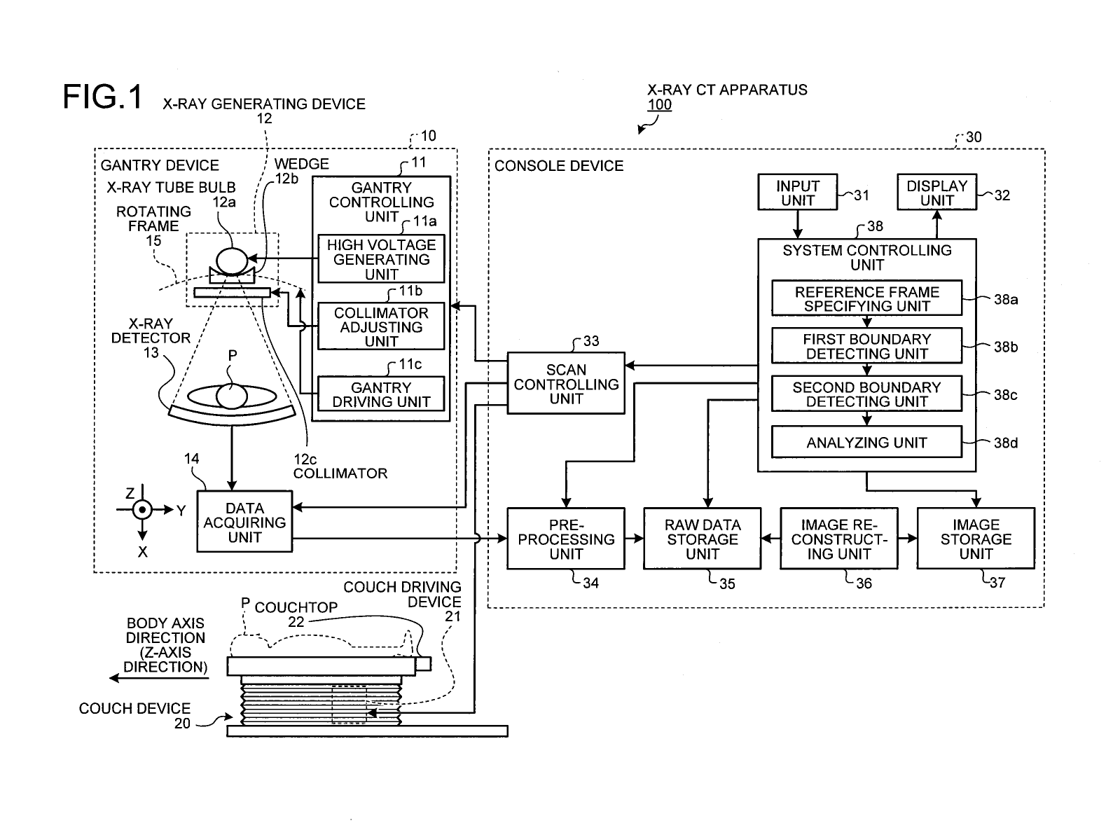

FIG. 1 is a diagram illustrating an X-ray CT apparatus 100 according to a first embodiment. As illustrated in FIG. 1, the X-ray CT apparatus 100 includes a gantry device 10, a couch device 20, and a console device 30 (which may be referred to as an “image processing apparatus”). Possible configurations of the X-ray CT apparatus 100 are not limited to those described in the exemplary embodiments below.

The gantry device 10 acquires projection data by radiating X-rays onto an examined subject (hereinafter, a “subject”) P. The gantry device 10 includes a gantry controlling unit 11, an X-ray generating device 12, an X-ray detector 13, a data acquiring unit 14, and a rotating frame 15.

Under control of a scan controlling unit 33 (explained later), the gantry controlling unit 11 controls operations of the X-ray generating device 12 and the rotating frame 15. The gantry controlling unit 11 includes a high voltage generating unit 11a, a collimator adjusting unit 11b, and a gantry driving unit 11c. The high voltage generating unit 11a supplies a high voltage to an X-ray tube bulb 12a. The collimator adjusting unit 11b adjusts the radiation range of the X-rays radiated onto the subject P from the X-ray generating device 12, by adjusting the opening degree and the position of a collimator 12c. For example, the collimator adjusting unit 11b radiates the X-rays onto the subject P using a reduced X-ray radiation range (a reduced cone angle), by adjusting the opening degree of the collimator 12c. The gantry driving unit 11c drives the rotating frame 15 to rotate. While frame 15 rotates, the X-ray generating device 12 and the X-ray detector 13 turn on a circular orbit centered on the subject P.

The X-ray generating device 12 radiates the X-rays onto the subject P. The X-ray generating device 12 includes the X-ray tube bulb 12a, a wedge 12b, and the collimator 12c. The X-ray tube bulb 12a is a vacuum tube generates an X-ray beam (a cone beam) that spreads in a cone shape or a pyramid shape along the body axis direction of the subject P, by using the high voltage supplied by the high voltage generating unit 11a. The X-ray tube bulb 12a radiates the cone beam onto the subject P, in conjunction with the rotation of the rotating frame 15. The wedge 12b is an X-ray filter used for adjusting the dose of the X-rays radiated from the X-ray tube bulb 12a. The collimator 12c is a slit used for, under control of the collimator adjusting unit 11b, narrowing the radiation range of the X-rays of which the dose has been adjusted by the wedge 12b.

The X-ray detector 13 is a multi-row detector (which may be referred to as a “multi-slice detector” or a “multi-detector-row detector”) that has a plurality of X-ray detecting elements arranged in a channel direction (a row direction) and in a slice direction (a column direction). The channel direction corresponds to the rotating direction of the rotating frame 15, whereas the slice direction corresponds to the body axis direction of the subject P. For example, the X-ray detector 13 has the detecting elements that are arranged in 916 rows along the row direction and in 320 columns along the column direction. The X-ray detector 13 detects, in a wide region, the X-rays that have passed through the subject P. The quantity of the detecting elements is not limited to this example. It is desirable to provide the detecting elements in such a quantity that is able to realize a scanned region by which both the upper end and the lower end of the heart are scanned in one conventional scan, so that it is possible to obtain seamless volume data of the entirety of the heart. For example, if large-sized detecting elements are used, the detecting elements may be arranged in 900 rows along the row direction and in 256 columns along the column direction. Alternatively, to obtain volume data of the entirety of the heart having a number of seams, detecting elements may be used in an even smaller quantity. It is acceptable to use a multiple-row detector in which detecting elements are arranged in 16 or 64 columns along the column direction. In that situation, a helical scan is performed to acquire data of the entirety of the heart.

The data acquiring unit 14 amplifies signals detected by the X-ray detector 13, to generate projection data by applying an Analog/Digital (A/D) conversion to the amplified signals, and to transmit the generated projection data to the console device 30. The data acquiring unit 14 may be referred to as a Data Acquisition System (DAS).

The rotating frame 15 is an annular frame supporting the X-ray generating device 12 and the X-ray detector 13 so as to face each other while the subject P is interposed therebetween. By the gantry driving unit 11c, the rotating frame 15 is caused to rotate on the circular orbit centered on the subject P at a high speed.

The couch device 20 includes a couch driving device 21 and a couchtop 22 and has the subject P placed thereon. The couch driving device 21, under the control of the scan controlling unit 33 (explained later), moves the subject P to the inside of the rotating frame 15, by moving the couchtop 22 on which the subject P is placed in the Z-axis direction.

The console device 30 receives an operation performed on the X-ray CT apparatus 100 by the operator and to generate a CT image indicating internal morphology of the subject P, from the projection data acquired by the gantry device 10. The console device 30 includes an input unit 31, a display unit 32, the scan controlling unit 33, a pre-processing unit 34, a raw data storage unit 35, an image reconstructing unit 36, an image storage unit 37, and a system controlling unit 38.

The input unit 31 is configured by using a mouse and/or a keyboard that are used by the operator of the X-ray CT apparatus 100 to input various types of instructions and various types of settings and transfers information about the instructions and the settings received from the operator to the system controlling unit 38. The display unit 32 is a monitor referred to by the operator and, under control of the system controlling unit 38, displays a CT image or the like for the operator and displays a Graphical User Interface (GUI) used for receiving the various types of settings from the operator via the input unit 31.

The scan controlling unit 33, under the control of the system controlling unit 38, controls operations of the gantry controlling unit 11, the data acquiring unit 14, and the couch driving device 21. More specifically, by controlling the gantry controlling unit 11, the scan controlling unit 33 causes the rotating frame 15 to rotate, causes the X-ray tube bulb 12a to radiate the X-rays, and adjusts the opening degree and the position of the collimator 12c, during an image taking process performed on the subject P. Further, under the control of the system controlling unit 38, the scan controlling unit 33 controls the amplifying process, the A/D conversion process, and the like performed by the data acquiring unit 14. Furthermore, under the control of the system controlling unit 38, the scan controlling unit 33 moves the couchtop 22 by controlling the couch driving device 21, during an image taking process performed on the subject P.

The pre-processing unit 34 generates raw data by performing correcting processes such as a logarithmic conversion, an offset correction, a sensitivity correction, a beam hardening correction, a scattered beam correction, and the like on the projection data generated by the data acquiring unit 14 and to store the generated raw data into the raw data storage unit 35.

The raw data storage unit 35 stores therein the raw data generated by the pre-processing unit 34 kept in correspondence with an electrocardiogram signal acquired from an electrocardiograph attached to the subject P. The image reconstructing unit 36 generates the CT image by reconstructing the raw data stored in the raw data storage unit 35. The image storage unit 37 stores therein the CT image reconstructed by the image reconstructing unit 36.

The system controlling unit 38 exercises overall control of the X-ray CT apparatus 100 by controlling operations of the gantry device 10, the couch device 20, and the console device 30. More specifically, by controlling the scan controlling unit 33, the system controlling unit 38 causes an electrocardiogram-synchronized scan to be executed and arranges the projection data to be acquired from the gantry device 10. Further, by controlling the pre-processing unit 34, the system controlling unit 38 causes the raw data to be generated from the projection data. Furthermore, the system controlling unit 38 exercises control so that the display unit 32 displays the raw data stored in the raw data storage unit 35 and the CT image stored in the image storage unit 37.

The raw data storage unit 35 and the image storage unit 37 described above may be realized by using a semiconductor memory element (e.g., a Random Access Memory (RAM), a flash memory), a hard disk, an optical disk, or the like. Further, the scan controlling unit 33, the pre-processing unit 34, the image reconstructing unit 36, and the system controlling unit 38 described above may be realized by using an integrated circuit such as an Application Specific Integrated Circuit (ASIC) or a Field Programmable Gate Array (FPGA), or an electronic circuit such as a Central Processing Unit (CPU) or a Micro Processing Unit (MPU).

Further, in the first embodiment, the electrocardiograph (not shown) is used during an image taking process performed on the subject P. The electrocardiograph includes an electrocardiograph electrode, an amplifier, and an A/D conversion path and amplifies, with the use of the amplifier, electrocardiogram waveform data sensed as en electric signal by the electrocardiograph electrode and to eliminate noise from the amplified signal, so as to convert the signal into a digital signal.

When the X-ray CT apparatus 100 according to the first embodiment has generated a group of frames corresponding to a plurality of heartbeat phases, by reconstructing acquired image data of the heart in correspondence with each of the heartbeat phases, the X-ray CT apparatus 100 specifies a reference frame (may also be referred to as a “corresponding frame”) from among the group of frames and starts a process of detecting a boundary from the specified reference frame. In this situation, the reference frame is a frame that is among the group of frames corresponding to the plurality of heartbeat phases and that corresponds to a specific heartbeat phase. Further, in the first embodiment, for the purpose of detecting the boundary of the heart with a high accuracy, a heartbeat phase in which movement amount of the heart is relatively small is used as the specific heartbeat phase. In this regard, the first embodiment will be explained by using the diastolic phase (the mid-diastolic phase, in particular), as the heartbeat phase in which movement amount of the heart is relatively small. Because of having a relatively long time length, the mid-diastolic phase is suitable to be used as a reference frame, also in this sense. Processes described herein are realized by constituent elements of the image reconstructing unit 36 and the system controlling unit 38.

As illustrated in FIG. 1, the system controlling unit 38 includes a reference frame specifying unit 38a, a first boundary detecting unit 38b, a second boundary detecting unit 38c, and an analyzing unit 38d. Processes performed by these units will be explained briefly. First, the image reconstructing unit 36 reconstructs the raw data of the heart stored in the raw data storage unit 35 in correspondence with each of the heartbeat phases, generates the group of frames corresponding to the plurality of heartbeat phases, and stores the generated group of frames into the image storage unit 37. Further, the reference frame specifying unit 38a specifies the reference frame corresponding to the specific heartbeat phase from among the group of frames stored in the image storage unit 37. The first boundary detecting unit 38b detects the boundary of the heart from the reference frame specified by the reference frame specifying unit 38a. The second boundary detecting unit 38c detects a boundary of the heart from each of the frames other than the reference frame, by using the boundary detected by the first boundary detecting unit 38b. The analyzing unit 38d performs an analysis by using the boundaries of the heart detected from the frames by the first boundary detecting unit 38b and the second boundary detecting unit 38c.

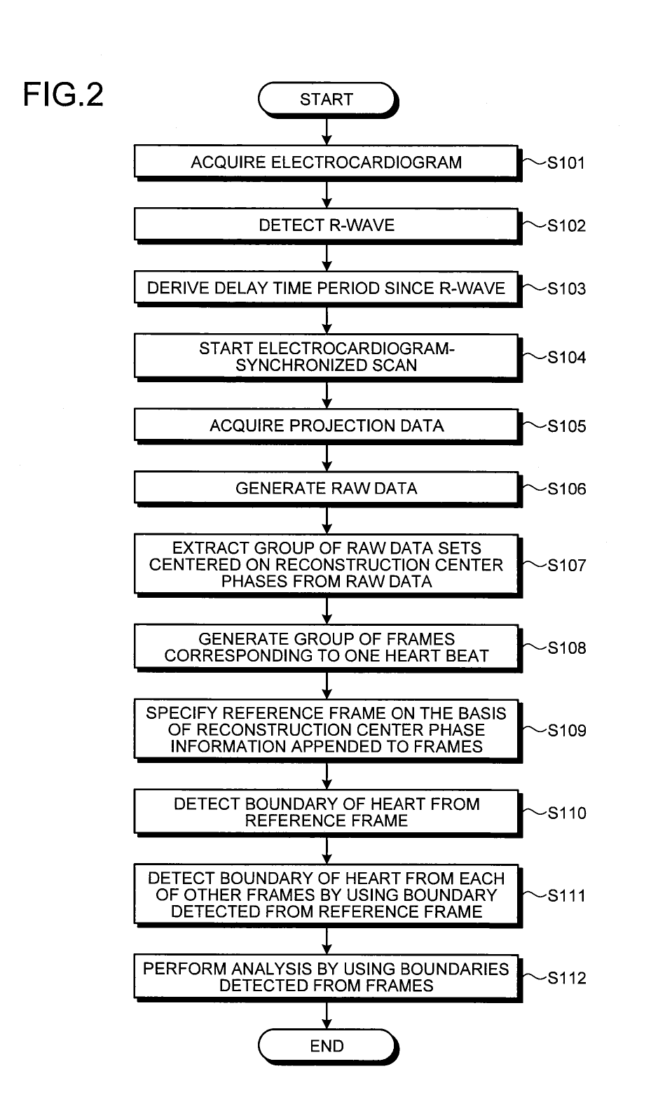

FIG. 2 is a flowchart of a processing procedure according to the first embodiment. The first embodiment is based on an example using a half reconstruction, as explained below; however, possible embodiments are not limited to this example. The disclosure herein is similarly applicable to a situation in which a full reconstruction is used or to a situation in which a segment reconstruction is used together. The processing procedure illustrated in FIG. 2 is explained in such a manner that the processing procedure for generating a group of frames from raw data and the processing procedure for specifying boundaries of the heart by specifying a reference frame from among the group of frames are processing procedures performed during a series of medical examination procedures; however, possible embodiments are not limited to this example. In another example, it is acceptable to perform the former processing procedure and the latter processing procedure on separate occasions.

In the first embodiment, at first, an electrocardiogram is acquired prior to an electrocardiogram-synchronized scan, for the purpose of deriving the timing with which an X-ray radiation is to be started during an electrocardiogram-synchronized scan, i.e., a delay time period since a characteristic wave (e.g., an R-wave) (step S101). In this situation, the electrocardiogram-synchronized scan is a method by which an electrocardiogram-synchronized signal (e.g., an R-wave signal) or an electrocardiogram waveform signal (e.g., an ECG signal) is acquired in parallel with a scan, so that an image is reconstructed in correspondence with each of the heartbeat phases by using the electrocardiogram signal such as the electrocardiogram-synchronized signal or the electrocardiogram waveform signal, after the data has been acquired. For example, the electrocardiograph is attached to the subject P, so that the electrocardiograph acquires the electrocardiogram signal of the subject P during a breathing practice time period when instructions such as “Please breathe in” and “Please hold your breath” are given and transmits the acquired electrocardiogram signal to the system controlling unit 38.

Subsequently, the system controlling unit 38 detects an R-wave from the received electrocardiogram signal (step S102), and after deriving an average interval corresponding to one heart beat (an R-R interval) during the breathing practice time period, the system controlling unit 38 derives a delay time period since the R-wave that serves as a trigger for starting an X-ray radiation, based on other conditions related to the scan (step S103). For example, other conditions related to the scan include a designation of an image taking site (e.g., the heart), an acquiring mode (e.g., 320 cross-sectional planes are acquired at the same time by using the detecting elements arranged in 320 columns), a heartbeat phase used as a target of the reconstruction, and a mode of the reconstruction (e.g., a half reconstruction).

After confirming that the electrocardiogram signal has been acquired by the electrocardiograph, the operator instructs that an electrocardiogram-synchronized scan should be started, so that the scan controlling unit 33 starts the scan under the control of the system controlling unit 38 (step S104). For example, the electrocardiogram signal of the subject P acquired by the electrocardiograph is transmitted to the system controlling unit 38, so that the system controlling unit 38 detects R-waves one after another from the received electrocardiogram signal. After that, based on the delay time period since the R-wave derived at step S103, the system controlling unit 38 transmits an X-ray control signal to the scan controlling unit 33. The scan controlling unit 33 acquires projection data of the heart, by controlling the X-ray radiation onto the subject P according to the received X-ray control signal (step S105).

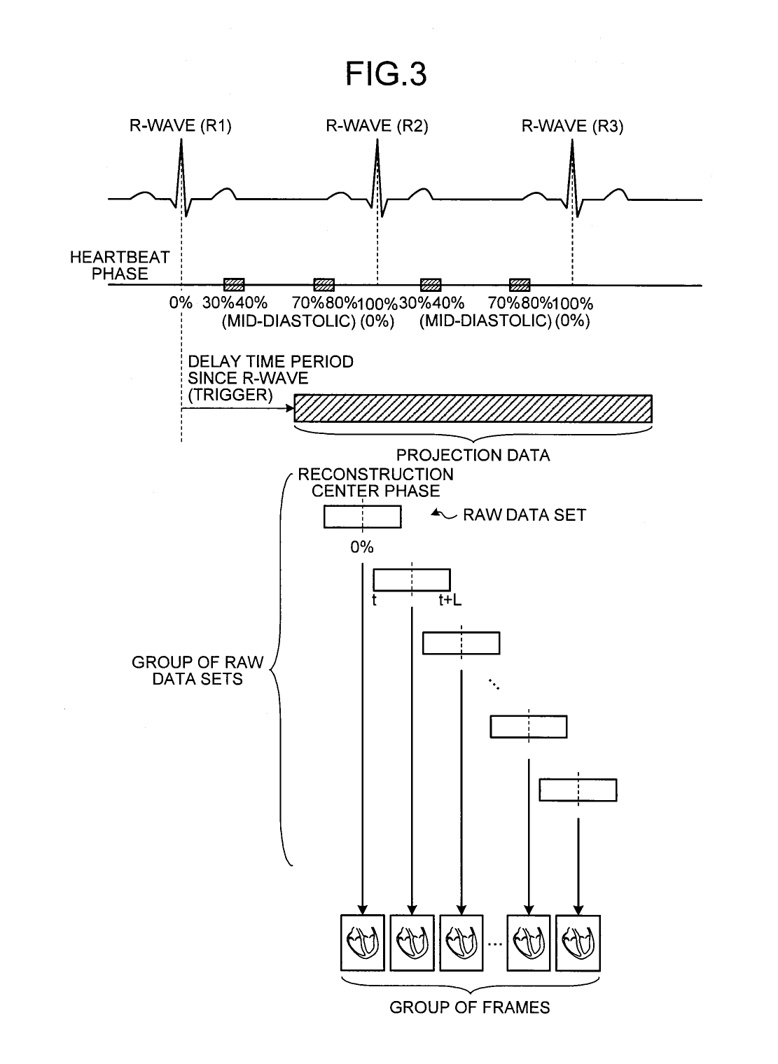

FIG. 3 is a drawing for explaining the generation of the group of frames according to the first embodiment. For example, as illustrated in FIG. 3, when the predetermined delay time period has elapsed since an R-wave (R1) serving as the trigger for starting the X-ray radiation, the scan controlling unit 33 starts the X-ray radiation and acquires the projection data. Further, as illustrated in FIG. 3, for example, the scan controlling unit 33 acquires projection data corresponding to one heart beat during (and before and after) the time period between the R-wave (R2) immediately following the R-wave (R1) serving as the trigger (R1) and the subsequent R-wave (R3), i.e., during one heart beat. In other words, in the first embodiment, because the X-ray detector 13 includes the detecting elements arranged in the 320 columns as described above, it is possible to acquire three-dimensional projection data of the entirety of the heart, by causing the rotating frame 15 to rotate once. Further, the rotating frame 15 acquires projection data used for reconstructing a plurality of heartbeat phases, by rotating three times during one heart beat, for example.

The pre-processing unit 34 applies various types of correcting processes to the three-dimensional projection data of the heart acquired in this manner, so that three-dimensional raw data of the heart is generated (step S106).

Subsequently, the image reconstructing unit 36 extracts a group of raw data sets from the raw data generated at step S106 (step S107), so as to generate a group of frames corresponding to the one heart beat, by using the extracted group of raw data sets (step S108). For example, when performing a half reconstruction, the image reconstructing unit 36 extracts, from the raw data, a raw data set acquired while the X-ray tube bulb 12a is rotating in the range of 180°+α (where α is the fan angle of fan-shaped X-rays), in such a manner that the raw data set is centered on each of a plurality of heartbeat phases designated by the operator (hereinafter, “reconstruction center phases”), for each of the reconstruction center phases. Subsequently, the image reconstructing unit 36 generates a group of raw data sets in the range of 360° from the extracted group of raw data sets, by using a two-dimensional filter that employs what is called a Parker's two-dimensional weight coefficient map. After that, the image reconstructing unit 36 generates a group of frames corresponding to a plurality of heartbeat phases by reconstructing the raw data sets contained in the generated group of raw data sets, by performing a back-projection process. The group of frames corresponding to the plurality of heartbeat phases is represented by volume data corresponding to each of the mutually-different cardiac phases and is represented by image data of three-dimensional images or multi-slice images (a plurality of tomographic images) corresponding to the mutually-different cardiac phases.

For example, as illustrated in FIG. 3, the image reconstructing unit 36 extracts, from the raw data, a raw data set for each of the reconstruction center phases and further generates a group of frames corresponding to the plurality of heartbeat phases from the group of raw data sets in the range of 360° generated from the extracted raw data sets. Each of the reconstruction center phases represents the position of the time period between an R-wave and the R-wave subsequent thereto and is expressed with “0-100%” or “milliseconds (msec)”. For example, when a cyclic period of one heart beat is divided into sections using 5% intervals, the reconstruction center phases are expressed as “0%”, “5%”, “10%”, . . . , “95%” and “100%”. The first embodiment is explained using the example in which the raw data sets are extracted from the raw data so as to be centered on the reconstruction center phases; however, possible embodiments are not limited to this example. In another example, each of the raw data sets in a predetermined range may be extracted while using a designated heartbeat phase as a starting point. In other words, the heartbeat phases used in the reconstruction do not necessarily have to be positioned at the center of the raw data sets, and may be in any arbitrary position.

In this situation, the image reconstructing unit 36 stores the generated group of frames into the image storage unit 37 using a data structure compliant with specifications of Digital Imaging and Communications in Medicine (DICOM). In the data structure compliant with the DICOM specifications, additional information is appended to image data. The additional information is an aggregate of data elements. Each of the data elements includes a tag and data corresponding to the tag. Further, a data type (a value representation) and a data length are defined for each of the data elements. Apparatuses that handle the data compliant with the DICOM specifications process the additional information according to the definitions. For example, the image reconstructing unit 36 appends the additional information to each of the frames, the additional information including reconstruction center phase information indicating the reconstruction center phase of the frame, as well as the name of the subject, the subject ID, the birth date (year, month, day) of the subject, the type of the medical image diagnosis apparatus used for acquiring the image data, a medical examination ID, a series ID, an image ID, and the like. For example, the tag of the reconstruction center phase information is appended as a private tag that is different from a standard tag. Further, possible embodiments are not limited to these examples. In another example, the image reconstructing unit 36 may append the reconstruction center phase information to each of the frames by using a format other than those that are compliant with the DICOM specifications.

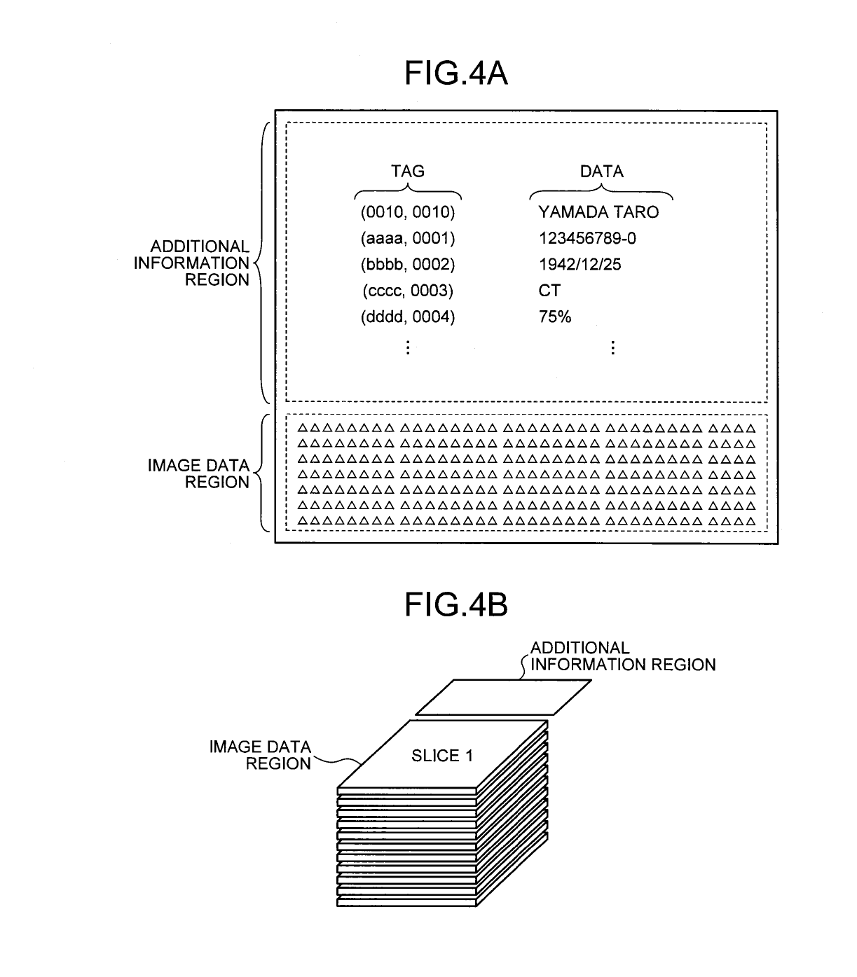

FIGS. 4A and 4B are drawings of frames compliant with the DICOM specifications according to the first embodiment. As illustrated in FIG. 4A, the data for each of the frames has an additional information region and an image data region. Further, the additional information region contains the data elements each of which is a set made up of a tag and data corresponding to the tag. In the example illustrated in FIG. 4A, for example, the tag (dddd, 0004) is a private tag of the reconstruction center phase information, whereas information indicating “75%” is contained as the data.

Further, FIG. 4A illustrates the data structure in which one piece of additional information (one additional information region) is appended to each piece of image data (a piece of single image data) corresponding to one slice. However, possible embodiments are not limited to this example. As illustrated in FIG. 4B, another data structure may be used in which one piece of additional information (one additional information region) that is shared among a plurality of slices is appended to image data (enhanced image data) corresponding to the plurality of slices. As explained above, the group of frames according to the first embodiment includes the pieces of volume data corresponding to the plurality of heartbeat phases, each piece of volume data corresponding to one heartbeat phase. In that situation, as illustrated in FIG. 4B, for example, a piece of volume data corresponding to one heartbeat phase contains image data corresponding to a plurality of slices. Thus, one piece of additional information (one additional information region) is appended to the image data corresponding to the plurality of slices.

Returning to the description of FIG. 2, when having read the group of frames stored in the image storage unit 37, the reference frame specifying unit 38a subsequently refers to the reconstruction center phase information appended to each of the frames and specifies a reference frame from among the group of frames (step S109). In this situation, according to the first embodiment, the reference frame specifying unit 38a specifies the reference frame corresponding to a heartbeat phase in which movement amount of the heart is relatively small, from among the group of frames. For example, as illustrated in FIG. 3, when a reconstruction center phase falls in the range from “30%” to “40%” or the range from “70%” to “80%”, the reconstruction center phase is considered to be a heartbeat phase in which movement amount of the heart is relatively small during the one heart beat. In that situation, from among the group of frames, the reference frame specifying unit 38a specifies the frame of which the reconstruction center phase information appended to the image data indicates “75%” (or a value closest to “75%”), for example, as the reference frame. In the first embodiment, it is assumed that the value “75%” is designated in advance. Further, when the reference frame specifying unit 38a is to specify a reference frame based on the heartbeat phase (e.g., “75%”) designated in advance, if there is no frame that corresponds to the heartbeat phase designated in advance, the reference frame specifying unit 38a specifies a frame corresponding to a heartbeat phase that is close to the heartbeat phase designated in advance (e.g., a value closest to “75%”), as the reference frame. Alternatively, the reference frame specifying unit 38a may use reconstruction center phase information designated at the time of the reconstruction, without using the DICOM additional information of the image data. In other words, when generating the group of frames corresponding to one heart beat by reconstructing the raw data as described above, the image reconstructing unit 36 extracts the group of raw data sets corresponding to the reconstruction center phases from the raw data and further generates the group of frames corresponding to the plurality of heartbeat phases by reconstructing each of the raw data sets. Thus, by appending the reconstruction center phase information to each of the frames by using a format other than those compliant with the DICOM specifications, the reference frame specifying unit 38a is able to specify a reference frame even if there is no DICOM additional information.

Returning to the description of FIG. 2, the first boundary detecting unit 38b subsequently detects a boundary of the heart from the reference frame specified at step S109 (step S110). In the first embodiment, the boundary of the heart is represented by the left ventricular epicardium, the right ventricular epicardium, the left atrial endocardium and epicardium, and the right atrial endocardium and epicardium. The first boundary detecting unit 38b is able to detect the boundary of the heart by using a publicly-known technique, for example. For example, because the lungs and blood are present in the surroundings of the boundary of the heart, the differences in the brightness levels between those and the boundary are known in advance. Accordingly, the first boundary detecting unit 38b is able to detect the boundary by dynamically changing the shape of a contour shape model obtained by statistically learning the hearts of a large number of subjects in advance, while using brightness level information of the surroundings of the boundary. As an initial shape of the contour shape model, the first boundary detecting unit 38b may use a shape obtained by changing an average heart shape resulting from a learning process performed in advance, according to the position and the orientation of the heart and a scale that are estimated separately. Further, the detected boundary of the heart is expressed by a plurality of control points.

After that, the second boundary detecting unit 38c detects a boundary of the heart from each of the frames in the group of frames other than the reference frame, by using the boundary detected at step S110 (step S111).

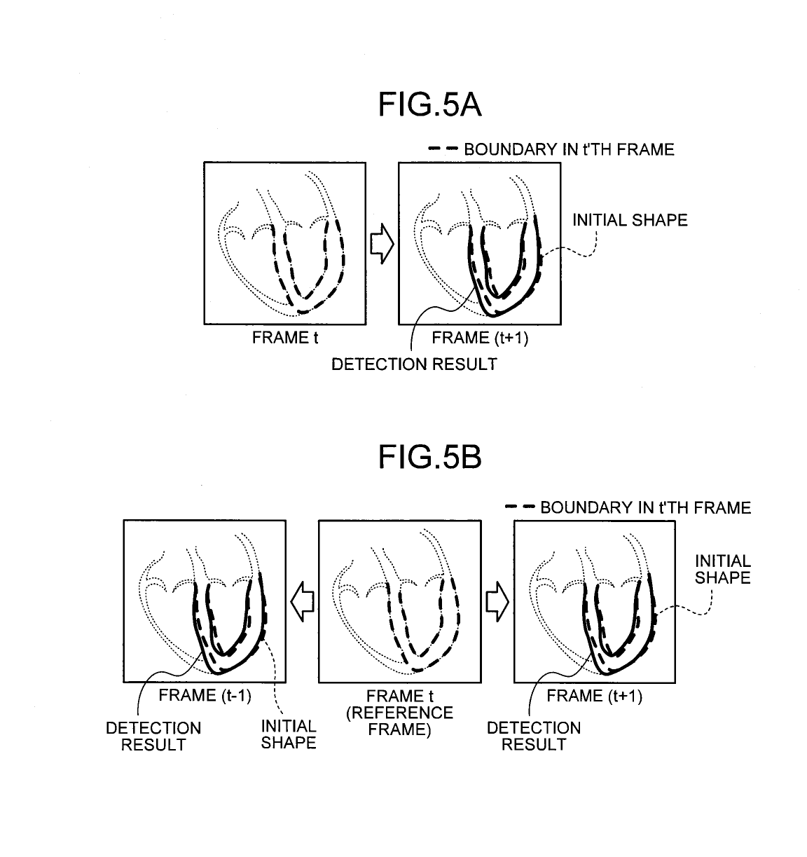

FIGS. 5A and 5B are drawings for explaining a boundary detecting process according to the first embodiment. For example, at first, the second boundary detecting unit 38c detects a boundary with respect to a frame (e.g., “frame t”) adjacent to the reference frame, by using the boundary detection result from the reference frame as an initial shape of the contour shape model. Subsequently, the second boundary detecting unit 38c detects a boundary with respect to the “frame (t+1)” adjacent to the “frame t”, by using the boundary detection result from the “frame t” as an initial shape of the contour shape model. In other words, the second boundary detecting unit 38c sequentially propagates a detection result from an adjacent frame, according to the order in the time series.

It is assumed that frames adjacent to each other (e.g., the “frame t” and the “frame (t+1)”) have heartbeat phases that are close to each other and have similar heart shapes. For this reason, when the detection result from the “t'th frame” is used as the initial shape of the contour shape model for the “(t+1)'th frame”, it is expected that the obtained initial shape has a higher accuracy than in the situation where an average contour shape model is used. The accuracy in the boundary detecting process using the dynamic contour shape model is dependent on the accuracy of the initial shape. Thus, by using the initial shape having a high accuracy, it is possible to reduce the number of times a repetitive calculation needs to be performed, and this feature also contributes to shortening the processing time. By sequentially applying the process described above to each of the frames following the reference frame, the second boundary detecting unit 38c detects a boundary from each of all the frames contained in the group of frames.

The boundary detecting process performed on the frames adjacent to each other does not necessarily have to be implemented by using the method described above. For example, the second boundary detecting unit 38c may detect a boundary in the “(t+1)'th frame” by estimating the positions to which a plurality of control points expressing the boundary in the “t'th frame” will move in the “(t+1)'th frame”, by performing a template matching process that employs an image pattern of the surroundings of the control points. In that situation, the image pattern may reflect information (e.g., brightness level information, brightness level gradient information, or the like) that is known in advance about the surroundings of the boundary of the heart.

Further, the boundary detecting process performed on the frames adjacent to each other does not necessarily have to be implemented by using the method described above. As illustrated in FIG. 5B, when the “t'th frame” is the reference frame, it is acceptable to propagate the detection result to the “(t−1)'th frame” and to the “(t+1)'th frame”, in both the normal order and the reverse order of the heartbeat phases.

After that, the analyzing unit 38d performs an analysis by using the boundaries of the heart detected from the frames at steps S110 and S111 (step S112). For example, the analyzing unit 38d analyzes the boundaries of the heart detected from the frames and calculates an Ejection Fraction (EF) value (i.e., a left ventricular ejection fraction) and/or the thickness of myocardia.

In the embodiments described above, the example is explained in which the electrocardiogram is acquired while the breathing practice is carried out, prior to the electrocardiogram-synchronized scan; however, possible embodiments are not limited to this example. In another example, the system controlling unit 38 may, after an electrocardiogram-synchronized scan has been started, derive a delay time period since the R-wave serving as the trigger for starting the X-ray radiation, by using an electrocardiogram signal obtained immediately before the X-ray radiation.

As explained above, according to the first embodiment, it is possible to, at first, improve the accuracy of the first detection by selecting the frame corresponding to the heartbeat phase in which movement amount of the heart is relatively small as the first frame used for boundary detecting process. As a result, it is possible to detect the boundary of the heart from each of all the frames with a high accuracy.

The first embodiment is explained above, by using the example in which the diastolic phase (the mid-diastolic phase, in particular) is used as the heartbeat phase in which movement amount of the heart is relatively small. Because of having a relatively long time length, the mid-diastolic phase is suitable to be used as a reference frame, also in this sense. Another reason for selecting the mid-diastolic phase is that images in the mid-diastolic phase are more likely to be selected as the images serving as the data to be learned.

This point will be explained further. It is desirable to select a frame that makes it possible to detect a boundary of the heart with a high accuracy, as the reference frame. For example, when the boundary detecting process is performed by using a dictionary that is learned in advance, it is assumed to be desirable to select, as the reference frame, an image acquired in the same heartbeat phase as the heartbeat phase in which the image used in the learning process was acquired. It is assumed that images of the heart acquired in mutually the same heartbeat phase have more similar shapes to each other than images of the heart acquired in mutually-different heartbeat phases. Thus, by performing the boundary detecting process while using the frame reconstructed in the heartbeat phase that is close to the heartbeat phase in which the image used in the learning process was acquired, it is possible to detect the boundary with a high accuracy.

For example, let us assume that it is often the case that an image in the mid-diastolic phase is acquired as a diagnosis-purpose image. In that situation, it is easy to acquire images in the mid-diastolic phase. Accordingly, the images in the mid-diastolic phase are used as the data to be learned for creating a dictionary, which requires a large number of samples for the purpose of detecting the boundary with a high accuracy. Consequently, it is desirable to also specify, as the reference frame, a frame that is reconstructed in the mid-diastolic heartbeat phase.

It should be noted, however, that the heartbeat phase specified as the reference frame does not necessarily have to be the mid-diastolic phase. It is acceptable to use any heartbeat phase as long as the movement amount of the heart is relatively small. For example, the end-diastolic phase or the end-systolic phase may be used. For example, if images acquired in the end-diastolic phase are used as learned data, it is acceptable to select the end-diastolic phase as the heartbeat phase for the reference frame.

When the end-diastolic phase is used as the heartbeat phase for the reference frame, for example, the reference frame specifying unit 38a may specify, as the reference frame, a frame of which the appended reconstruction center phase information indicates “0%” (or a value closest to “n”), for example. Because the heartbeat phases are set based on the relative positions of the R-R intervals in the electrocardiogram signal, the heartbeat phase corresponding to “0%” is near the end-diastolic phase.

### Modification Examples of the First Embodiment

In the first embodiment described above, the method is explained by which the reference frame is specified based on the reconstruction center phase information appended to each of the frames; however, possible embodiments are not limited to this example.

In another example, if the end-diastolic phase is used as the heartbeat phase for a reference frame and if an electrocardiogram signal is appended to the group of frames, the reference frame specifying unit 38a may specify a frame acquired during a certain time period extending before and after an R-wave used as a reference point, as a reference frame acquired in the end-diastolic phase. Also, when the mid-diastolic phase is used as the heartbeat phase for a reference frame, the reference frame specifying unit 38a may specify a frame acquired during a certain time period selected by using an R-wave as a reference point. Further, in yet example, the reference frame specifying unit 38a may specify a reference frame based on characteristics of images. For example, the reference frame specifying unit 38a may estimate a scale of the heart in each of all the frames by using a publicly-known technique. Scales of the heart have a correlation with heartbeat phases. (For example, the scale is larger in the diastolic phase, whereas the scale is smaller in the systolic phase). Thus, if the end-diastolic phase is used as the heartbeat phase for a reference frame, the reference frame specifying unit 38a may specify a frame of which the estimated scale of the heart is the largest. To estimate the scales of the heart, three-dimensional images may be used, or two-dimensional cross-sectional images may be used. Further, the example in which the boundary detecting process is performed by using the dictionary learned in advance is explained above; however, the learned data may be used in the reference frame specifying process itself. For example, the reference frame specifying unit 38a may specify a reference frame by performing a pattern matching process between the learned data from the end-diastolic phase and the frames in the group of frames. In any of these various types of methods explained as modification examples, any heartbeat phase in which movement amount of the heart is relatively small can be selected as the reference frame. Thus, possible modification examples are not limited to those described above.

Like in the exemplary embodiment described above, the X-ray CT apparatus 100 according to a second embodiment specifies a reference frame from among the group of frames and to start the heart boundary detecting process with the reference frame. In the first embodiment described above, the example is explained in which the frame corresponding to the predetermined reconstruction center phase is specified as the reference frame, by using the additional information appended to each of the frames; however, possible embodiments are not limited to this example. The X-ray CT apparatus 100 according to the second embodiment calculates movement amounts of the heart in heartbeat phases by analyzing the frames (or sinogram data) and specifies a reference frame by specifying a frame having a relatively small movement amount of the heart based on the result of the calculation.

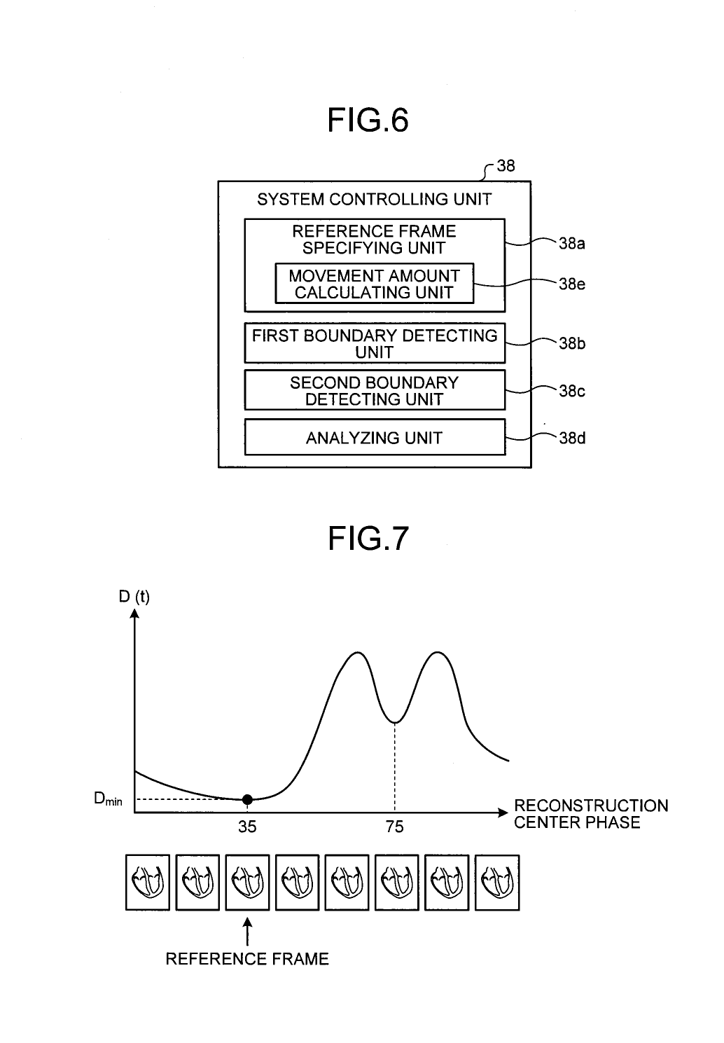

FIG. 6 is a diagram of the system controlling unit 38 according to the second embodiment. As illustrated in FIG. 6, in the second embodiment, the reference frame specifying unit 38a further includes a movement amount calculating unit 38e.

The movement amount calculating unit 38e calculates the movement amounts of the heart over the plurality of heartbeat phases by analyzing the frames stored in the image storage unit 37 (or the sinogram data stored in the raw data storage unit 35). For example, the movement amount calculating unit 38e calculates a movement amount of the heart by calculating a difference “D(t)” in pixel values between frames that are adjacent to each other according to the order in the time series and that are among the group of frames generated by the image reconstructing unit 36.

FIG. 7 is a drawing for explaining a reference frame specifying process according to the second embodiment. When the movement amounts of the heart calculated by the movement amount calculating unit 38e is plotted, while the movement amount “D(t)” of the heart is expressed on the vertical axis, whereas the reconstruction center phase is expressed on the horizontal axis, a time-based change rate curve as illustrated in FIG. 7, for example, is obtained.

Accordingly, of the time-based change rate curve, for example, the reference frame specifying unit 38a specifies the reconstruction center phase (e.g., “35” in FIG. 7) in which movement amount of the heart is relatively smallest and specifies a reference frame by specifying the frame reconstructed in the specified reconstruction center phase.

The movement amount calculation performed by the movement amount calculating unit 38e does not necessarily have to be implemented by using the method described above. For example, the movement amount calculating unit 38e may calculate the movement amounts of the heart over the plurality of heartbeat phases, by analyzing the sinogram data stored in the raw data storage unit 35. This method has a lighter processing load than the method by which the frames are analyzed. Thus, the processing time is expected to be shortened.

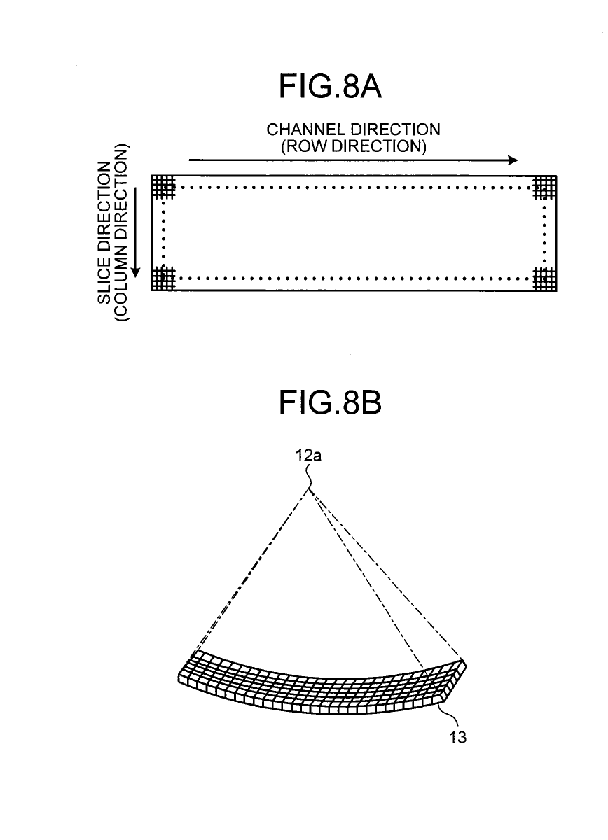

FIGS. 8A and 8B are drawings for explaining the X-ray detector 13 according to the second embodiment. FIG. 8A is a top view of the X-ray detector 13. As illustrated in FIG. 8A, for example, the X-ray detector 13 includes detecting elements that are arranged in 916 rows along the channel direction (the row direction) and in 320 columns along the slice direction (the column direction). FIG. 8B is a perspective view.

The signal detected by the X-ray detector 13 configured as described above is subsequently generated into projection data by the data acquiring unit 14 and is further generated into raw data by the pre-processing unit 34. The sinogram data is a locus of the brightness level of the projection data that is plotted while the view (the position of the X-ray tube bulb 12a) is expressed on the vertical axis, whereas the channel is expressed on the horizontal axis.

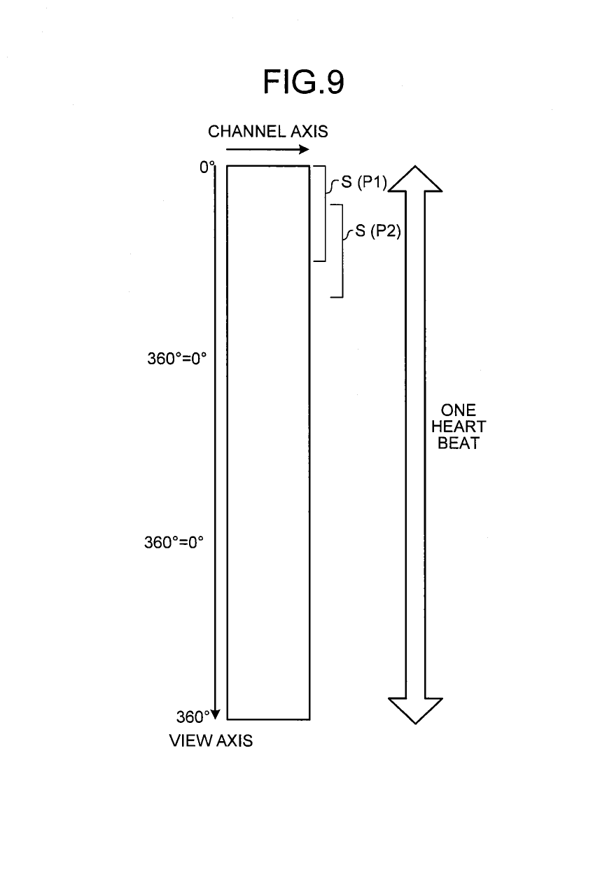

FIG. 9 is a drawing for explaining the reference frame specifying process according to the second embodiment. For example, in the second embodiment, let us discuss a situation in which the rotating frame 15 rotates three times in one heart beat so as to acquire projection data used for reconstructing a plurality of heartbeat phases. In this situation, it is assumed that the sinogram data is structured so that, as illustrated in FIG. 9, the view expressed on the vertical axis corresponds to three turns each containing 0°-360°. The sinogram data illustrated in FIG. 9 is sinogram data structuring a certain column, i.e., a specific cross-sectional plane. Sinogram data such as that illustrated in FIG. 9 is available for each of the 320 columns, for example. A cross-sectional plane rendering the left ventricle may be used as the specific cross-sectional plane, for example. Further, a locus of the brightness level of the projection data is omitted from FIG. 9.

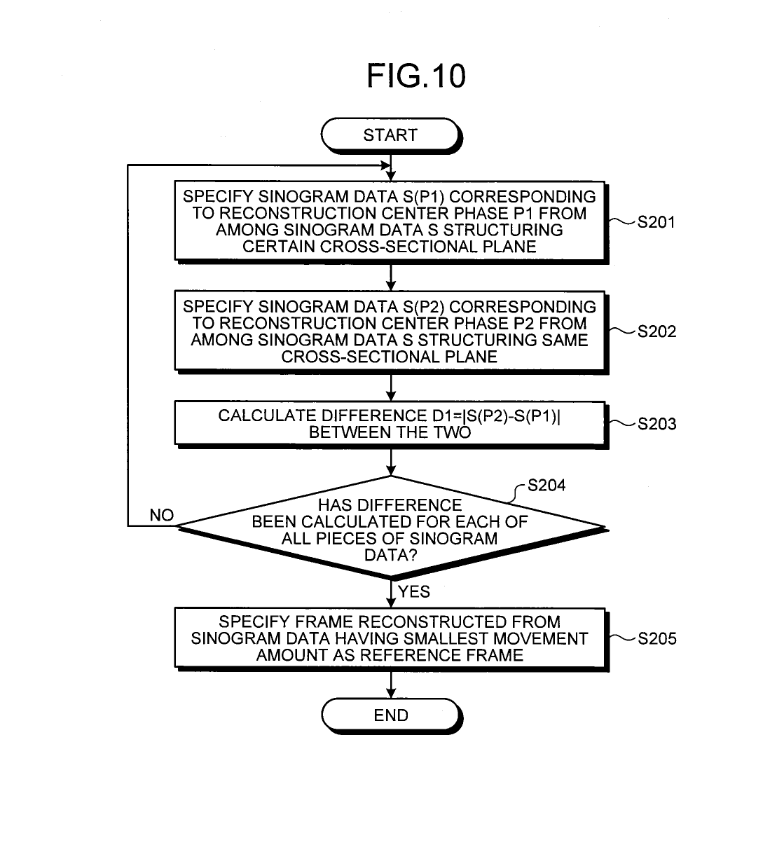

FIG. 10 is a flowchart of a processing procedure in the reference frame specifying process according to the second embodiment. First, the movement amount calculating unit 38e specifies sinogram data S(P1) corresponding to a reconstruction center phase P1, from among sinogram data S structuring a certain cross-sectional plane (step S201). Further, from among the sinogram data S structuring the same cross-sectional plane, the movement amount calculating unit 38e specifies sinogram data S(P2) corresponding to a reconstruction center phase P2 that is adjacent to the reconstruction center phase P1 according to the order in the time series (step S202).

Subsequently, the movement amount calculating unit 38e calculates the difference D1 between S(P2) and S(P1) (step S203). After that, the movement amount calculating unit 38e judges whether a difference has been calculated for each of all the pieces of sinogram data (step S204). If the difference calculation has not been completed for all the pieces of sinogram data (step S204: No), the movement amount calculating unit 38e repeatedly performs the processes at steps S201 through S203, by shifting the reconstruction center phase specified at steps S201 and S202. On the contrary, if the difference calculation has been completed for all the pieces of sinogram data (step S204: Yes), the reference frame specifying unit 38a specifies a piece of sinogram data having the relatively smallest difference D based on the calculation results. After that, the reference frame specifying unit 38a specifies a frame reconstructed from the specified piece of sinogram data as a reference frame (step S205). When there is a movement of the heart, there supposed to be a difference in the sinogram data. This method therefore places a focus on this difference.

The example illustrated in FIG. 10 is explained by using the sinogram data structuring a certain cross-sectional plane (a certain column); however, possible embodiments are not limited to this example. In another example, it is also acceptable to use sinogram data corresponding to a plurality of cross-sectional planes (a plurality of columns) in a range that is able to cover the heart. Further, with reference to FIG. 10, the example is explained in which each of the differences is calculated between the reconstruction center phases that are adjacent to each other; however, possible embodiments are not limited to this example. The interval of the reconstruction center phases to be compared with each other may be arbitrarily determined.

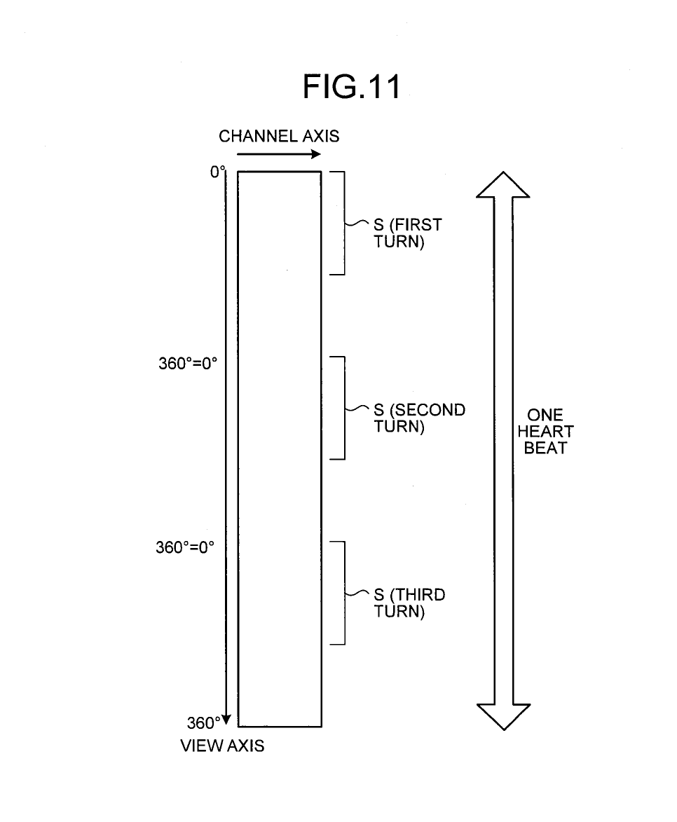

Further, in yet another example, it is acceptable to calculate a difference between pieces of sinogram data of which the positions of the views (i.e., the positions of the X-ray tube bulb 12a) are the same. FIG. 11 is a drawing for explaining another reference frame specifying process according to the second embodiment. For example, as illustrated in FIG. 11, the movement amount calculating unit 38e may calculate differences by comparing sinogram data S (for the first turn) “from 0° to (180°+α) of the first turn”, sinogram data S (for the second turn) “from 0° to (180°+α) of the second turn”, and sinogram data S (for the third turn) “from 0° to (180°+α) of the third turn”.

For example, if the reconstruction center phases of these three pieces of sinogram data are “0%”, “35%”, and “75%”, the reference frame specifying unit 38a compares, for example, the difference between “0%” and “35%” with the difference between “35%” and “75%”. The reference frame specifying unit 38a then determines that the pair having the smaller difference has a relatively smaller movement amount of the heart. Consequently, for example, the reference frame specifying unit 38a specifies a frame reconstructed from the sinogram data of which the reconstruction center phase is at “75%”, as a reference frame.

In FIG. 11, the sinogram data is assumed to be sinogram data of which the view width ranges from 0° to (180°+α); however, possible embodiments are not limited to this example. It is acceptable to use sinogram data having a smaller view width.

As explained above, according to the second embodiment, the reference frame is specified by analyzing the frames (or the sinogram data). Thus, the reference frame is specified based on the data actually acquired. Consequently, the accuracy with which the reference frame is specified is improved. As a result, it is possible to detect the boundary of the heart in each of all the frames with a higher accuracy.

Like in the exemplary embodiments described above, the X-ray CT apparatus 100 according to a third embodiment specifies a reference frame from among the group of frames and to start the heart boundary detecting process with the reference frame. In the exemplary embodiments described above, the example is explained in which the reconstruction center phases used for reconstructing the frames are designated in advance; however, possible embodiments are not limited to this example. In the third embodiment, the reconstruction center phases themselves are specified by analyzing the sinogram data.

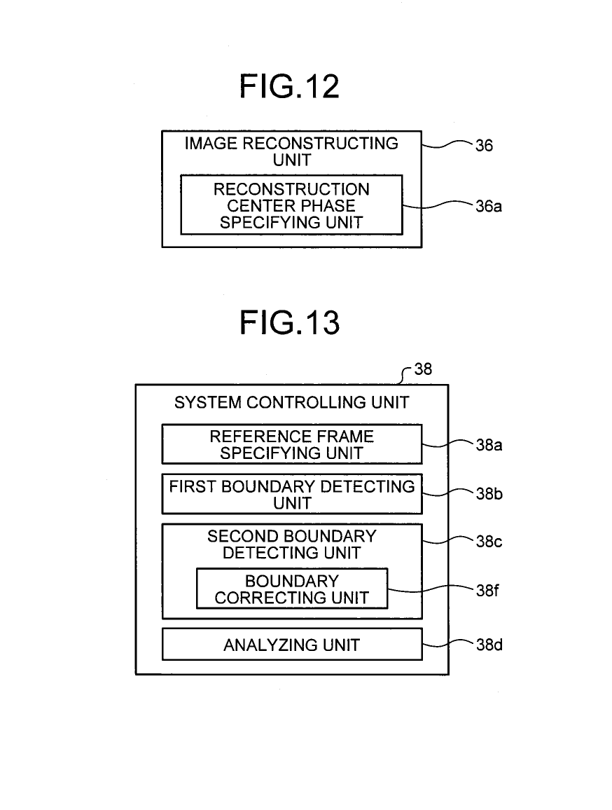

FIG. 12 is a diagram of the image reconstructing unit 36 according to the third embodiment. As illustrated in FIG. 12, in the third embodiment, the image reconstructing unit 36 further includes a reconstruction center phase specifying unit 36a. For example, the reconstruction center phase specifying unit 36a calculates movement amounts of the heart in heartbeat phases by analyzing the sinogram data stored in the raw data storage unit 35 by implementing the method explained in the second embodiment, for example, and specifies a heartbeat phase in which movement amount of the heart is relatively smallest.

For example, when the difference D is calculated between heartbeat phases, it is possible to specify reconstruction center phases in units that are smaller than the intervals (e.g., 5% intervals) of the reconstruction center phases designated in advance, by decreasing the intervals between the heartbeat phases to be compared with each other. For example, even if the reconstruction center phase at “75%” is designated according to the intervals of the reconstruction center phases designated in advance, it is possible to specify reconstruction center phases in smaller units such as “72%” or “79%” in the third embodiment. Further, the reconstruction center phase specifying unit 36a specifies such a heartbeat phase as the reconstruction center phase for the first frame, for example. For the other frames, the reconstruction center phase specifying unit 36a may set the reconstruction center phases as appropriate, for example, by setting the reconstruction center phases at 5% intervals, while using the reconstruction center phase for the reference frame as a starting point.

When the reconstruction center phases are specified in this manner, it is expected that a desired image (e.g., an image in the mid-diastolic phase in which movement amount of the heart is relatively smallest) is obtained as the first frame with a higher accuracy.

In the third embodiment, the example is explained in which the reconstruction center phase specifying unit 36a uses the analysis result of the sinogram data for the purpose of specifying the reconstruction center phase for the first frame, for example; however, possible embodiments are not limited to this example. In another example, the reconstruction center phase specifying unit 36a may use the analysis result of the sinogram data for the purpose of determining sections in which the frame reconstruction is to be performed during one heart beat. For example, let us discuss a situation in which the analysis performed by the analyzing unit 38d is to obtain the thickness of myocardia, and it is sufficient if frames in the end-systolic phase and the end-diastolic phase are reconstructed. In that situation, for example, the reconstruction center phase specifying unit 36a specifies the actual heartbeat phases corresponding to the end-systolic phase and the end-diastolic phase, by using the analysis result of the sinogram data. Further, the image reconstructing unit 36 may reconstruct the frames only in the sections of the heartbeat phases specified by the reconstruction center phase specifying unit 36a.

As explained above, according to the third embodiment, the reconstruction center phases themselves are specified by analyzing the frames (or the sinogram data). Because the frames are reconstructed based on the reconstruction center phases that are specified from the data actually acquired, it is expected possible to further improve the accuracy with which the boundary is detected from the reference frame. As a result, it is possible to detect the boundary of the heart from each of all the frames with a higher accuracy.

Like in the exemplary embodiments described above, the X-ray CT apparatus 100 according to a fourth embodiment specifies a reference frame from among the group of frames and to start the heart boundary detecting process with the reference frame. Further, the X-ray CT apparatus 100 according to the fourth embodiment displays, in a superimposed manner, the boundaries of the heart detected from the frames and the images in the frames and to receive a correction instruction from the operator.

FIG. 13 is a diagram of the system controlling unit 38 according to the fourth embodiment. As illustrated in FIG. 13, the second boundary detecting unit 38c further includes a boundary correcting unit 38f. The boundary correcting unit 38f causes the display unit 32 to display the boundaries of the heart superimposed on the image in the frames, the superimposed boundaries are detected from the frames. The boundary correcting unit 38f receives the correction instruction from the operator. Further, when having received the correction instruction, the boundary correcting unit 38f re-detects a boundary of the heart from the frame for which the correction instruction was received.

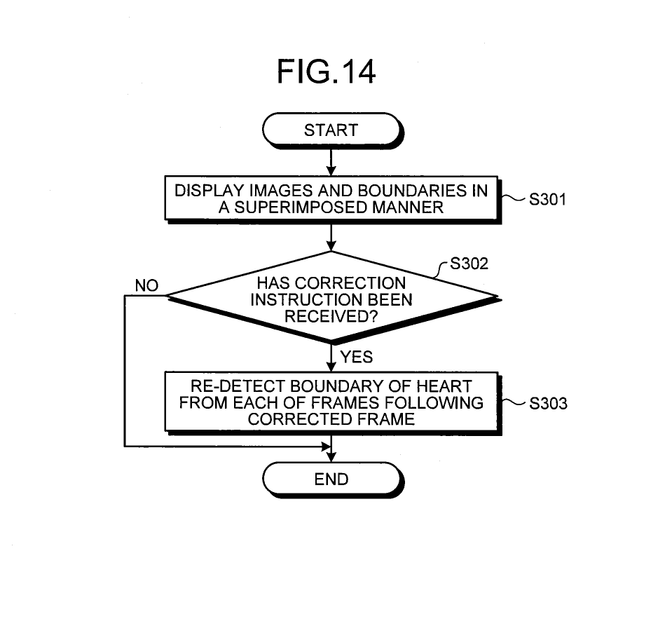

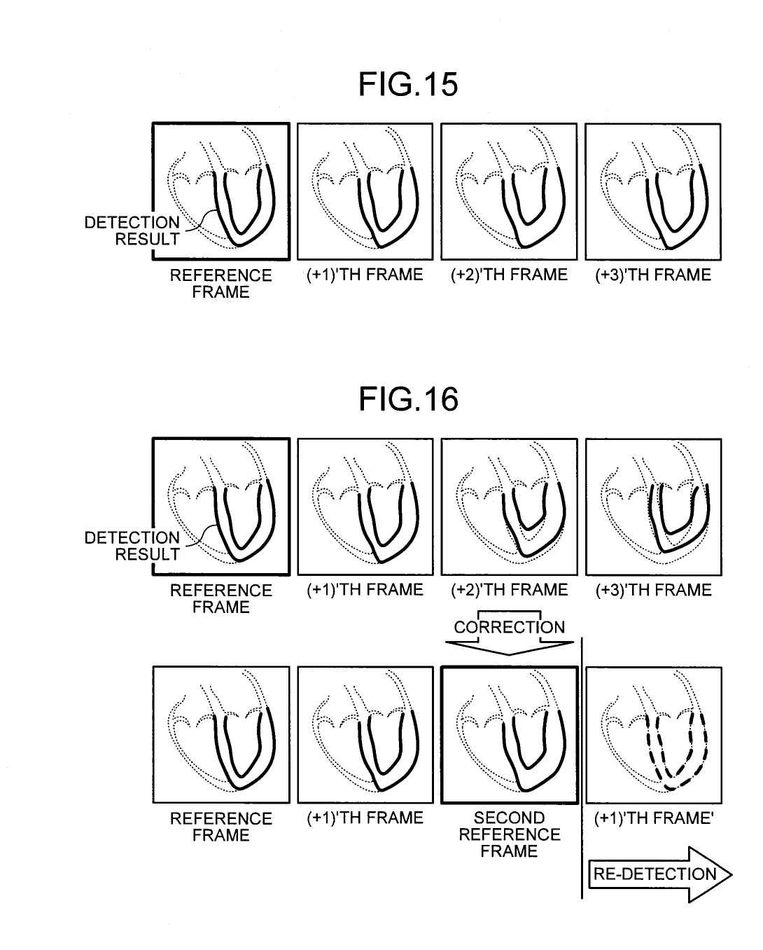

FIG. 14 is a flowchart of a processing procedure in a boundary correcting process according to the fourth embodiment. FIGS. 15 and 16 are drawings for explaining the boundary correcting process according to the fourth embodiment. For example, the processing procedure shown in FIG. 14 may be performed between steps S111 and S112 in the processing procedure shown in FIG. 2 in the first embodiment.

For example, with respect one or more frames, the boundary correcting unit 38f causes the display unit 32 to display, in a superimposed manner, the images in the frames and the boundaries of the heart temporarily detected from the frames (step S301). In this situation, for example, as illustrated in FIG. 15, the boundary correcting unit 38f displays the frames arranged in the order of heartbeat phases, while distinguishing between the reference frame and the other frames. Examples of methods for distinguishing between the frames include a method by which the colors of the borders of the images are varied and a method by which the names of the frames are clearly written (e.g., “reference frame” is clearly written for the reference frame).

Subsequently, the boundary correcting unit 38f judges whether a correction instruction has been received from the operator (step S302). For example, the operator looks at the superimposed display of the images and the boundaries displayed on the display unit 32 and corrects the boundary in such a frame from which the boundary was detected earliest after the reference frame, from among the frames each of which requires a correction. For example, the operator inputs a correction on the boundary via the input unit 31 that is configured with a pointing device such as a trackball. The operator may input a corrected boundary in a free-hand manner or may input a correction by adding, deleting, and/or moving the control points of the detected boundary. When the correction is made on a two-dimensional cross-sectional plane, the operator is able to arbitrarily change the cross-sectional plane that is displayed for the correction purpose. Alternatively, the image displayed for the correction purpose may be an image expressed in a three-dimensional manner.

Alternatively, the boundary correcting unit 38f may present a plurality of boundary candidates to the operator and prompt the operator to select one of the boundary candidates. For example, in the exemplary embodiments described above, the example is explained in which the first boundary detecting unit 38b and the second boundary detecting unit 38c detect the boundaries of the heart by using the contour shape model. However, for example, the first boundary detecting unit 38b and the second boundary detecting unit 38c are able to obtain a plurality of detection results by performing the same process after preparing a plurality of initial shape models. In that situation, for example, the boundary correcting unit 38f displays a detection result having the smallest error as a final detection result, by using evaluation values, such as an error calculated between an image pattern near the control points and an image pattern obtained from a learning process performed in advance or an error calculated between the shape of the detected boundary and a contour shape model obtained from a learning process performed in advance. Further, by causing the display unit 32 to display the other detection results as the candidates for the boundary correction purposes, the boundary correcting unit 38f presents the boundary candidates to the operator.

When the operator has input the correction in this manner, the boundary correcting unit 38f determines that a correction instruction has been received (step S302: Yes) and further re-detects a boundary from each of the frames following a second reference frame, which is the frame in which the boundary was corrected by the operator (step S303). For example, as illustrated in FIG. 16, if the boundary correcting unit 38f has determined that a correction instruction has been received with respect to the “(+2)'th frame”, the boundary correcting unit 38f uses the “(+2)'th frame” as the second reference frame and re-detects a boundary from each of the frames, namely the “(+3)'th frame” and thereafter. After the boundary correcting unit 38f has re-detected the boundary at step S303, the process returns to step S301 where the re-detection result is presented to the operator.

As explained with reference to FIGS. 4A and 4B, the boundary detecting process is performed by using the detecting result of the immediately preceding frame. Thus, if the detection fails in one frame, the error is propagated to the other frames thereafter, and there is a possibility that the detection may not be performed correctly. For this reason, it is desirable to re-detect a boundary from each of the frames following the frame in which the boundary was corrected. Further, by automatically detecting the boundary in each of the frames following the frame corrected by the operator, it is possible to keep cumbersome boundary correcting operations to a minimum. Thus, this feature contributes to improving the efficiency of diagnosis processes.

As explained above, according to the fourth embodiment, the operator is able to detect the boundary of the heart from each of all the frames with a higher accuracy, by performing only a small number of correcting operations.

Like in the exemplary embodiments described above, the X-ray CT apparatus 100 according to a fifth embodiment specifies a reference frame from among the group of frames and to start the heart boundary detecting process with the reference frame. Further, the X-ray CT apparatus 100 according to the fifth embodiment calculates a deviation amount between the reference frame and each of the other frames and to specify one or more frames serving as an analysis target based on the calculated deviation amounts.

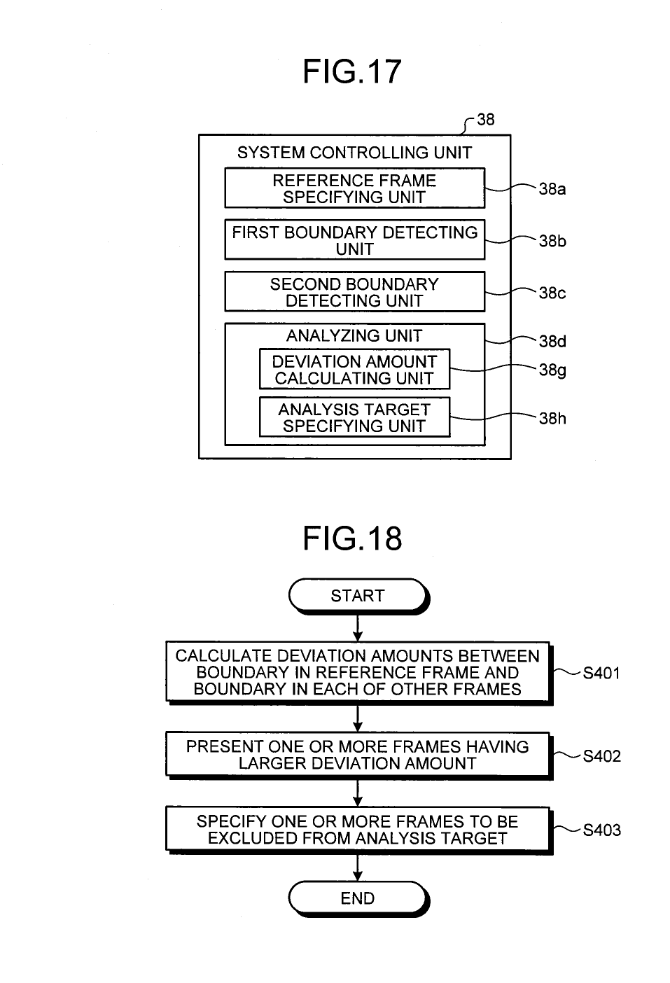

FIG. 17 is a diagram of the system controlling unit 38 according to the fifth embodiment. As illustrated in FIG. 17, in the fifth embodiment, the analyzing unit 38d further includes a deviation amount calculating unit 38g and an analysis target specifying unit 38h. The deviation amount calculating unit 38g calculates the deviation amount between the reference frame and each of the frames other than the reference frame and to cause the display unit 32 to display the calculation results. The analysis target specifying unit 38h specifies one or more frames serving as an analysis target or one or more frames to be excluded from the analysis target by receiving a designation from the operator, the designation being made from among the group of frames and indicating the one or more frames serving as the analysis target or the one or more frames to be excluded from the analysis target.

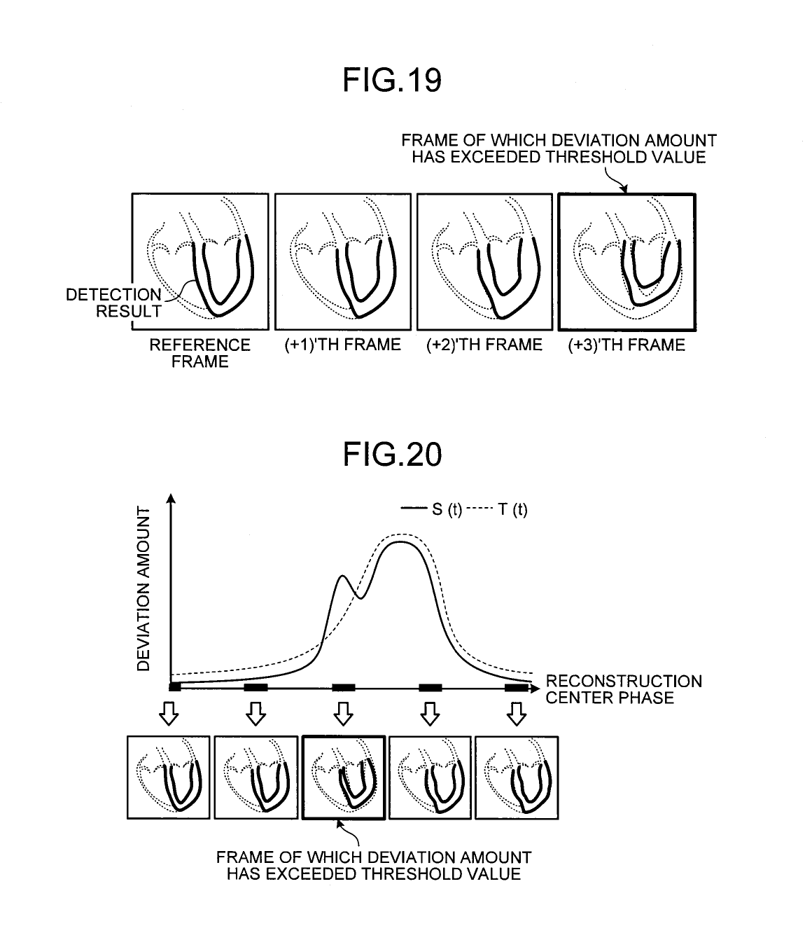

FIG. 18 is a flowchart of a processing procedure in an analysis target specifying process according to the fifth embodiment. FIGS. 19 and 20 are drawings for explaining the analysis target specifying process according to the fifth embodiment. For example, the processing procedure shown in FIG. 18 may be executed before the analysis performed at step S112 in the processing procedure shown in FIG. 2 in the first embodiment.

For example, the deviation amount calculating unit 38g calculates boundary deviation amounts by calculating the difference between the boundary in the reference frame detected by the first boundary detecting unit 38b and the boundary in each of the other frames detected by the second boundary detecting unit 38c (step S401). For example, when each of the boundaries is expressed by a set of control points on the boundary, the deviation amount calculating unit 38g calculates a deviation amount S(t) in the boundaries between the reference frame and a t'th frame, by using Expression (1) shown below:

$$\begin{matrix}S ( t ) = {\sum }_{i = 1}^{N} {( {X}_{i}^{0} - {X}_{i}^{t} )}^{T} A ( {X}_{i}^{0} - {X}_{i}^{t} ) & ( 1 ) \\ ( \begin{matrix}{X}_{i}^{0} \text{:} The boundary in the reference frame \\ {X}_{i}^{t} \text{:} An {i}^{′} th control point representing the boundary in {t}^{′} th frame \\ A \text{:} A normalized matrix\end{matrix} ) & \end{matrix}$$

In this situation, the normalized matrix A is set in advance. If the normalized matrix A is an identity matrix, the deviation amount S(t) is expressed as a squared Euclidean distance, whereas if the normalized matrix A is an inverse matrix of a covariance matrix, the deviation amount S(t) is expressed as a squared Mahalanobis distance. It should be noted that the deviation amount is not limited to the sum of squared errors at the mutually-different points, which is expressed in Expression (1). In another example, the deviation amount may be any index that expresses the difference in the boundaries between two frames, such as the sum of absolute-value errors, the sum of distances between a control point and another control point, or the sum of distances between each of the control points and the boundary. To obtain the sum of distances between a control point and another control point, a distance between a control point in the t'th frame and the corresponding control point in the (t+1)'th frame is calculated, so as to calculate the sum of such distances for all the control points. To obtain the sum of distances between each of the control points and the boundary, the boundary is expressed with a curve calculated from the control points by performing a spline interpolation process or the like, so as to calculate the distance between a control point in the t'th frame and a point that is positioned on the boundary in the (t+1)'th frame and is positioned closest to the control point in the t'th frame and to further calculate the sum of such distances for all the control points.

If the deviation amount of the boundary calculated from the t'th frame exhibits a value larger than a deviation amount caused by a movement or a deformation of the heart, there is a possibility that the boundary detection may have failed in the frame. Thus, by calculating the deviation amounts from the boundary detected from the reference frame in the manner described above, it is possible to determine whether the boundary detecting process in the t'th frame has been successful or not.

Subsequently, the deviation amount calculating unit 38g presents, to the operator, one or more frames of which the calculated boundary deviation amount has exceeded a predetermined threshold value (step S402). For example, the deviation amount calculating unit 38g calculates, in advance, an average deviation amount S_{E}(t) and a standard deviation σ(t) of a frame that is in the same heartbeat phase as that of the t'th frame and further sets a threshold value T(t) so as to satisfy T(t)=S_{E}(t)+σ(t). After that, the deviation amount calculating unit 38g compares the deviation amount calculated at step S401 with the threshold value and displays one or more frames of which the calculated boundary deviation amount has exceeded the threshold value, while distinguishing between the one or more frames and the other frames. For example, as illustrated in FIG. 19, the deviation amount calculating unit 38g displays the frames arranged in the order of heartbeat phases, while distinguishing between the reference frame and the other frames and also distinguishing between the frame of which the deviation amount has exceeded the threshold value and the other frames. Examples of methods for distinguishing between the frames include a method by which the colors of the borders of the images are varied and a method by which the names of the frames are clearly written. Further, as illustrated in FIG. 20, for example, the deviation amount calculating unit 38g may cause the display unit 32 to display changes in the deviation amount S(t) and the threshold value T(t), together with the group of frames.

Subsequently, the analysis target specifying unit 38h specifies one or more frames to be excluded from the analysis target (step S403). For example, the analysis target specifying unit 38h specifies the one or more frames to be excluded from the analysis target by prompting the operator to designate which frames should be excluded from the analysis target. Alternatively, for example, the analysis target specifying unit 38h may prompt the operator to designate one or more frames that are not to be excluded from the analysis target. In this situation, for example, the analysis target specifying unit 38h may automatically specify the one or more frames of which the deviation amount has exceeded the threshold value according to the calculation result obtained at step S401, as the frames to be excluded from the analysis target. In that situation, the presenting process at step S402 may be omitted. Because there is a possibility that the boundary detection may have failed in such a frame that has a large deviation amount, it is possible to obtain an analysis result (e.g., a function analysis result) having high reliability by excluding such a frame from the analysis process performed by the analyzing unit 38d.

Further, in the fifth embodiment, the example is explained in which the one or more frames to be excluded from the analysis target are specified after the deviation amounts are displayed; however, possible embodiments are not limited to this example. In another example, it is acceptable to simply end the process when the deviation amount calculating unit 38g has displayed the deviation amounts.

As explained above, according to the fifth embodiment, it is possible to obtain a heart analysis result having high reliability.

### Other Embodiments

Possible embodiments are not limited to the exemplary embodiments described above. The disclosure herein may be carried out in other various modes.

Specifying a Reference Frame by Using the Raw Data

In the second embodiment described above, the method is explained by which the movement amounts of the heart are calculated by analyzing the sinogram data, so that the frame reconstructed from the piece of sinogram data having the smallest movement amount is specified as the reference frame. Further, in the third embodiment, the method is explained by which the reconstruction center phases are specified by analyzing the sinogram data. However, possible embodiments are not limited to these examples. It is possible to specify a reference frame or to specify reconstruction center phases, by analyzing the raw data.

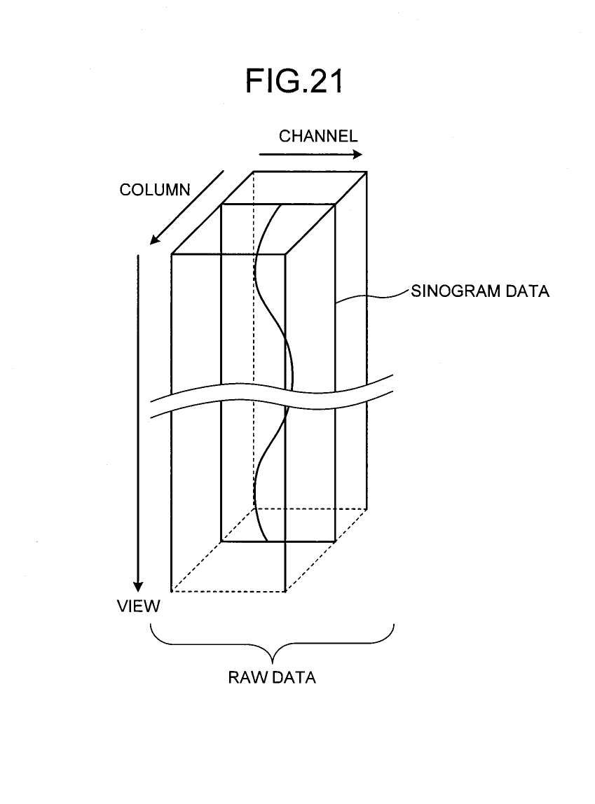

FIG. 21 is a drawing for explaining the raw data in an exemplary embodiment. A relationship between the raw data and the sinogram data will be briefly explained, with reference to FIG. 21. As explained in the second embodiment, the sinogram data is a locus of the brightness level of the projection data that is plotted while the view (the position of the X-ray tube bulb 12a) is expressed on the vertical axis, whereas the channel is expressed on the horizontal axis. Further, as illustrated in FIG. 21, usually, a range that structures one column (i.e., a specific cross-sectional plane) is referred to as sinogram data. In contrast, the raw data is generated by applying a pre-processing process to the entirety of the three-dimensional projection data, for example, and the range thereof corresponds to the entirety of the sinogram data corresponding to a plurality of columns. In other words, the sinogram data is one method for expressing the raw data.

For example, by analyzing the raw data, the movement amount calculating unit 38e calculates movement amounts of the heart in heartbeat phases. For example, from among the raw data stored in the raw data storage unit 35, the movement amount calculating unit 38e specifies raw data (R1) corresponding to a certain reconstruction center phase (P1). Further, the movement amount calculating unit 38e specifies raw data (R2) corresponding to a reconstruction center phase P2 that is adjacent to the reconstruction center phase P1 according to the order in the time series. The movement amount calculating unit 38e performs a process of calculating the difference between the raw data (R1) and the raw data (R2) while shifting the reconstruction phase. The reference frame specifying unit 38a then specifies a piece of raw data having the relatively smallest difference, based on the calculation results. After that, the reference frame specifying unit 38a specifies a frame reconstructed from the specified piece of raw data as a reference frame. When there is a movement of the heart, there supposed to be a difference also in the raw data. This method therefore places a focus on this difference. Similarly, when calculating a difference between heartbeat phases while using the pieces of raw data as comparison targets, the reconstruction center phase specifying unit 36a is able to specify reconstruction center phases in smaller units by decreasing the intervals between the heartbeat phases to be compared with each other.

Methods for Directly Specifying a Reference Frame

In the exemplary embodiments described above, the example is primarily explained in which the reference frame is specified after the heartbeat phase is specified, for example, by specifying the frame in the mid-diastolic heartbeat phase (e.g., “75%”) as the reference frame; however, possible embodiments are not limited to this example. The reference frame specifying unit 38a may directly specify a frame, a piece of raw data, or a piece of sinogram data having a relatively small movement amount of the heart, from among the group of frames being stored in the image storage unit 37 and corresponding to the plurality of heartbeat phases or from among the raw data or the sinogram data being stored in the raw data storage unit 35 and corresponding to the plurality of heartbeat phases. In other words, the reference frame specifying unit 38a does not necessarily have to specify a heartbeat phase when specifying a reference frame. The reference frame specifying unit 38a may specify a reference frame by specifying, for example, a frame having a relatively small movement amount of the heart (which may also be expressed as a frame having a stable contour shape of the heart). For example, the reference frame specifying unit 38a may perform an image analysis on each of the frames included in the group of frames, specify a frame having a relatively small movement amount of the heart according to results of the image analysis, and may use the specified frame as a reference frame.

Learning the Reference Frame

In the embodiments described above, the examples are primarily explained in which the frame in the heartbeat phase set in advance is specified as the reference frame and in which the reference frame is specified by specifying a frame having a relatively small movement amount of the heart. However, in actuality, there may be some situations where the reference frame specified in these manners is not necessarily an optimal reference frame. In those situations, the operator may correct the selection of the reference frame itself, for example.

In one example, at the stage when a reference frame has been specified, the reference frame specifying unit 38a may present the reference frame to the operator, prompt the operator to visually check the reference frame, and receive a reference frame change instruction. In another example, at the stage when the second boundary detecting unit 38c has temporarily detected the boundaries of the heart, the reference frame specifying unit 38a may present the boundary detection result and the reference frame to the operator, prompt the operator to visually check the boundary detection result and the reference frame, and to receive a reference frame change instruction. In yet another example, at the stage when the analyzing unit 38d performs the analysis, the reference frame specifying unit 38a may present the reference frame to the operator, prompt the operator to visually check the reference frame, and receive a reference frame change instruction.

When the reference frame itself is changed in an ex post facto manner as described above, for example, the reference frame specifying unit 38a may learn the reference frame resulting from the change (hereinafter, a “reference frame after the change”) and arrange the reference frame specifying process performed thereafter to reflect what is learned. In other words, when the reference frame specifying unit 38a has received the change instruction to change the specified reference frame from the operator, the reference frame specifying unit 38a stores therein and learns the reference frame after the change, while the first boundary detecting unit 38b proceeds with the process of newly detecting a boundary from the reference frame after the change. After that, the reference frame specifying unit 38a specifies a new reference frame according to the stored reference frame after the change. For example, if it has been determined in advance that, as an initial value, a frame in the mid-diastolic heartbeat phase (e.g., “75%”) is to be specified as a reference frame, after the reference frame specifying unit 38a has learned a number of times that the reconstruction center phase of a reference frame after a change is “80%”, the reference frame specifying unit 38a eventually changes the process so as to specify a frame at “80%” as a reference frame.

### Exemplary Embodiments in Combination

The exemplary embodiments described above may be carried out in combination, as appropriate. For example, in the first embodiment, the method is explained by which the reference frame is specified based on the reconstruction center phase information appended to each of the frames. Further, in the second embodiment, for example, the method is explained by which the movement amounts of the heart are calculated by analyzing the frames or the sinogram data so as to specify the reference frame based on the calculation results. Further, in the third embodiment, for example, the method is explained by which the reconstruction center phases themselves used for the reconstruction are specified by analyzing the sinogram data. Further, in the fourth embodiment, for example, the method is explained by which the boundaries of the heart detected from the heart are corrected. Furthermore, in the fifth embodiment, for example, the method is explained by which the deviation amounts in the boundaries between the reference frame and each of the other frames are calculated, so that the one or more frames to be excluded from the analysis target are specified based on the calculation results. All or a part of the description of any of the exemplary embodiments may be carried out individually or in combination. For example, by using the first embodiment and the second embodiment in combination, it is possible to complement one of the reference frame specifying methods with the other reference frame specifying method (e.g., to select one having the higher reliability).

Helical Scan and Step-and-Shoot Process

In the exemplary embodiments described above, the acquisition mode is explained in which the X-ray CT apparatus 100 includes the X-ray detector 13 having the detecting elements arranged in the 320 columns, so as to simultaneously detect the signals corresponding to the 320 cross-sectional planes. In this configuration, the X-ray CT apparatus 100 is normally able to simultaneously acquire the raw data in the range covering the entirety of the heart; however, possible embodiments are not limited to this example. In another example, the X-ray CT apparatus 100 may acquire raw data by using an acquisition mode called a helical scan or a step-and-shoot process. The helical scan is a method by which the subject P is helically scanned, by continuously moving the couchtop 22 on which the subject P is placed with a predetermined pitch along the body axis direction, while the rotating frame 15 is continuously rotating. The step-and-shoot process is a method by which the subject P is scanned, by moving the couchtop 22 on which the subject P is placed along the body axis direction in stages. When a helical scan or a step-and-shoot process is performed, projection data corresponding to one heart beat may be acquired during a plurality of heart beats, in some situations. In those situations, the X-ray CT apparatus 100 may obtain projection data corresponding to each of the reconstruction center phases, by gathering and combining the pieces of projection data corresponding to the plurality of mutually-different heart beats.

Application to Data Other than the Three-Dimensional Data

In the exemplary embodiments described above, the example is explained in which the X-ray CT apparatus 100 acquires the three-dimensional raw data and uses the acquired raw data as the processing target; however, possible embodiments are not limited to this example. The disclosure herein is similarly applicable to a situation where two-dimensional raw data is acquired. Further, in the exemplary embodiments described above, the example is explained in which the first boundary detecting unit 38b and the second boundary detecting unit 38c detect the boundaries of the heart from the group of three-dimensional frames; however, possible embodiments are not limited to this example. In another example, the first boundary detecting unit 38b and the second boundary detecting unit 38c may generate a group of cross-sectional images (e.g., Multi-Planar Reconstruction [MPR] images) that are suitable for the heart boundary detecting process from a group of three-dimensional frames and may further detect boundaries of the heart from the generated group of cross-sectional planes.

Application to a Magnetic Resonance Imaging (MRI) Apparatus

In the exemplary embodiments described above, the example using the X-ray CT apparatus as the medical image diagnosis apparatus is explained; however, possible embodiments are not limited to this example. For instance, it is possible to similarly apply the exemplary embodiments described above to an MRI apparatus. For example, the MRI apparatus may acquire Magnetic Resonance (MR) signals by applying a Radio Frequency (RF) pulse or a gradient magnetic field to the subject P after a predetermined delay time period has elapsed since an R-wave serving as a trigger and to obtain k-space data used for reconstructing images by arranging the acquired MR signals into a k-space. In view of time resolutions, the MRI apparatus, for example, divides k-space data corresponding to images in one heartbeat phase into a plurality of segments and acquires pieces of segment data during a plurality of mutually-different heart beats. In that situation, the MRI apparatus acquires segment data corresponding to a plurality of heartbeat phases during one heart beat. Further, the MRI apparatus gathers pieces of segment data that are in mutually the same heartbeat phase and each of which is acquired during a different one of the plurality of mutually-different heart beats, to arrange the gathered pieces of segment data into one k-space, and to reconstruct images corresponding to one heartbeat phase from the k-space data. Even in this example with the MRI apparatus, if heartbeat phase information is appended to each of the frames reconstructed from the pieces of k-space data, it is possible to specify a reference frame having a relatively small movement amount of the heart based on the heartbeat phase information. In the example using the MRI apparatus, it is possible to generate data having information that is the same as or similar to that of the sinogram data described in the exemplary embodiments above, by applying a one-dimensional Fourier transform to the k-space data.

Application to an Image Processing Apparatus

In the exemplary embodiments described above, the example is explained in which the X-ray CT apparatus executes the processes of specifying the reference frame, detecting the boundaries, and performing the analysis; however, possible embodiments are not limited to this example. Alternatively, an image processing apparatus that is different from the medical image diagnosis apparatus or an image processing system including the medical image diagnosis apparatus and an image processing apparatus may execute the various types of processes explained above. In this situation, the image processing apparatus may be configured with, for example, a workstation (a viewer), an image server of a Picture Archiving and Communication System (PACS), or any of various types of apparatuses used in an electronic medical record system. For example, the X-ray CT apparatus executes up to the process of generating the frames and appends the reconstruction center phase information, a medical examination ID, a subject ID, a series ID, and the like to the generated frames according to the DICOM specifications. Further, the X-ray CT apparatus stores the frames to which the various types of information are appended into the image server. Further, for example, the workstation is configured so that an analysis application is activated so as to calculate an Ejection Fraction (EF) value (i.e., a left ventricular ejection fraction) or a thickness of a myocardium and reads a corresponding group of frames from the image server, by providing the image server with a designation of a medical examination ID, a subject ID, a series ID, and the like at the time when the analysis is started, for example. Because the reconstruction center phase information is appended to the group of frames, the workstation is able to specify a reference frame and to perform the processes thereafter, based on the appended reconstruction center phase information. The image processing apparatus or the image processing system is also able to execute the other processes explained in the exemplary embodiments above. The information (e.g., the sinogram data) required during the processes may be transferred from the medical image diagnosis apparatus to the image processing apparatus or to the image processing system as appropriate, either directly or via the image server or via a storage medium (e.g., a Compact Disk [CD], a Digital Versatile Disk [DVD], a network storage).

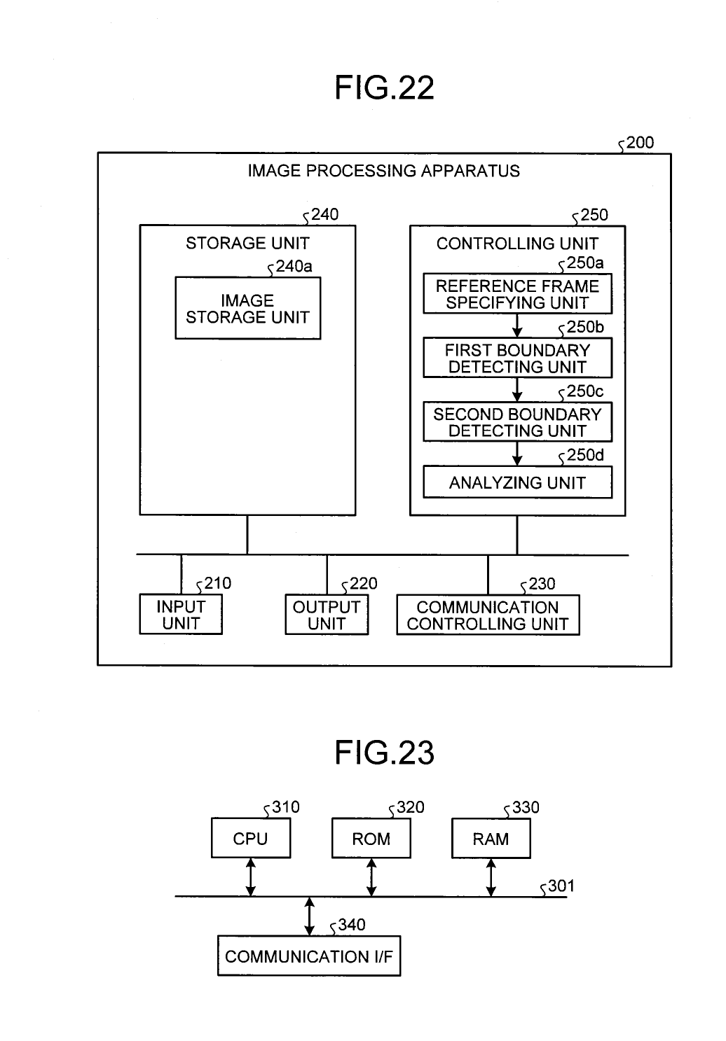

FIG. 22 is a diagram of an image processing apparatus 200 according to an exemplary embodiment. For example, the image processing apparatus 200 includes an input unit 210, an output unit 220, a communication controlling unit 230, a storage unit 240, and a controlling unit 250. The input unit 210, the output unit 220, the image storage unit 240a of the storage unit 240, and the controlling unit 250 correspond to the input unit 31, the display unit 32, the image storage unit 37, and the system controlling unit 38 included in the console device 30 illustrated in FIG. 1, respectively. Further, the communication controlling unit 230 is an interface which communicates with the image server and the like. Further, the controlling unit 250 includes a reference frame specifying unit 250a, a first boundary specifying unit 250b, a second boundary specifying unit 250c, and an analyzing unit 250d. These units correspond to the reference frame specifying unit 38a, the first boundary detecting unit 38b, the second boundary detecting unit 38c, and the analyzing unit 38d included in the console device 30 illustrated in FIG. 1, respectively. Further, the image processing apparatus 200 may further include a unit that corresponds to the image reconstructing unit 36.

Computer Program

The various types of processes described above may be realized by, for example, using a generally-used computer as basic hardware. For example, it is possible to realize the reference frame specifying unit 38a, the first boundary detecting unit 38b, the second boundary detecting unit 38c, and the analyzing unit 38d described above, by causing a processor installed in a computer to execute a computer program (hereinafter, a “program”). The various types of processes may be realized by installing the program into the computer in advance or by storing the program into a storage medium such as a CD or distributing the program via a network and subsequently installing the program into the computer as appropriate.

Others

The processing procedures, the names, the various types of parameters, and the like explained in the exemplary embodiments above may arbitrarily be altered unless noted otherwise. For example, in the exemplary embodiments described above, the example is explained in which the single frame is specified as the reference frame; however, possible embodiments are not limited to this example. It is acceptable to specify a plurality of frames as reference frames. For example, the reference frame specifying unit 38a may specify two frames at “35%” and “75%” as the reference frames, which are the frames corresponding to reconstruction center phases each having a relatively small movement amount of the heart. In that situation, the boundary detecting process performed by the second boundary detecting unit 38c may be started by using these two frames as starting points. Further, in the exemplary embodiments described above, the example using the X-ray detector 13 having the detecting elements arranged in the 320 columns along the column direction is explained; however, possible embodiments are not limited to this example. In other examples, the quantity of columns may be any arbitrary value such as 84, 128, or 160. The same applies to the quantity of rows.

Hardware Configuration

FIG. 23 is a diagram of a hardware configuration of an image processing apparatus according to any of the exemplary embodiments. The image processing apparatus according to any of the exemplary embodiments described above includes: a controlling device such as a Central Processing Unit (CPU) 310; storage devices such as a Read-Only Memory (ROM) 320 and a Random Access Memory (RAM) 330; a communication interface (I/F) 340 performs communication while being connected to a network; and a bus 301 connecting the constituent elements to one another.

The program executed by the image processing apparatus according to any of the exemplary embodiments described above is provided as being incorporated in the ROM 320 or the like in advance. Alternatively, it is also acceptable to provide the program executed by the image processing apparatus according to any of the exemplary embodiments described above by recording the program as a file in an installable or executable format, on a computer-readable recording medium such as a Compact Disk Read-Only Memory (CD-ROM), a Flexible Disk (FD), a Compact Disk Recordable (CD-R), or a Digital Versatile Disk (DVD), so as to provide the program as a computer program product.

Alternatively, it is also acceptable to provide the program executed by the image processing apparatus according to any of the exemplary embodiments described above by storing the program in a computer connected to a network such as the Internet and having the program downloaded via the network. Alternatively, it is also acceptable to provide or distribute the program executed by the image processing apparatus according to any of the exemplary embodiments described above, via a network such as the Internet.

The program executed by the image processing apparatus according to any of the exemplary embodiments described above may be realized by causing a computer to function as the constituent elements (e.g., the image reconstructing unit 36, the reference frame specifying unit 38a, the first boundary detecting unit 38b, the second boundary detecting unit 38c, and the analyzing unit 38d, as well as the reference frame specifying unit 250a, the first boundary specifying unit 250b, the second boundary specifying unit 250c, and the analyzing unit 250d) of the image processing apparatus described above. The computer is configured so that the CPU 310 is able to read the program from a computer-readable storage medium into a main storage device and to execute the read program.

By using the image processing apparatus and the X-ray CT apparatus according to at least one aspect of the exemplary embodiments described above, it is possible to detect the boundaries of the heart with a high accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An image processing apparatus comprising:
a generator that generates a group of frames corresponding to reconstructed images that correspond to a plurality of heartbeat phases of a heart;
a selector that specifies a corresponding frame that corresponds to a specific heartbeat phase from among the group of frames;
a first detector that detects a boundary of the heart in the corresponding frame; and
a second detector that detects a boundary of the heart in the frames other than the corresponding frame, by using the detected boundary in the corresponding frame.

2. The apparatus according to claim 1, wherein the selector specifies the corresponding frame that corresponds to a heartbeat phase in which movement amount of the heart is relatively small, from among the group of frames.

3. The apparatus according to claim 1, wherein the selector specifies the corresponding frame according to the specific heartbeat phase that is preliminary designated by an operator.

4. The apparatus according to claim 1, wherein the selector calculates movement amounts of the heart in heartbeat phases and specifies the corresponding frame based on the movement amounts of the heart.

5. The apparatus according to claim 4, wherein the selector calculates the movement amounts of the heart from acquired image data and specifies a frame that is obtained by reconstructing the acquired image data in a range that includes a heartbeat phase in which movement amount of the heart is relatively small as the corresponding frame.

6. The apparatus according to claim 1, wherein the second detector further causes a display unit to display the boundaries of the heart superimposed on the image in the frames, the superimposed boundaries are detected from the frames and receives a correction instruction from an operator.

7. The apparatus according to claim 6, wherein the second detector receives, as the correction instruction, an operation to correct the detected boundary in one of the frames displayed on the display unit, sets the frame in which the detected boundary is corrected as a second corresponding frame, and re-detects a boundary of the heart in each of frames generated after the second corresponding frame by using the corrected boundary in the second corresponding frame.

8. The apparatus according to claim 1, further comprising: an analyzer, wherein
the analyzer includes:
a calculator that calculates deviation amounts in the boundaries between the corresponding frame and each of the frames other than the corresponding frame and to cause a display unit to display a result of the calculation; and
a target selector that specifies one or more frames serving as an analysis target, by receiving a designation being made from among the group of frames and indicating the one or more frames serving as the analysis target or one or more frames to be excluded from the analysis target.

9. The apparatus according to claim 1, further comprising: an analyzer, wherein
the analyzer includes:
a calculator that calculates deviation amounts in the boundaries between the corresponding frame and each of the frames other than the corresponding frame; and
a target selector that specifies one or more frames serving as an analysis target from among the group of frames, based on the deviation amounts in the boundaries.

10. The apparatus according to claim 2, wherein the selector specifies the corresponding frame based on Digital Imaging and Communications in Medicine appended to each of pieces of image data included in the group of frames.

11. The apparatus according to claim 2, wherein the selector specifies the corresponding frame based on reconstruction center phase information designated at a time of a reconstruction.

12. The apparatus according to claim 1, wherein the selector specifies the corresponding frame that corresponds to a mid-diastolic phase among heartbeat phases.

13. The apparatus according to claim 1, wherein, when the selector is to specify the corresponding frame from among the group of frames based on the heartbeat phase designated in advance, if there is no frame that corresponds to the heartbeat phase designated in advance, the selector specifies a frame corresponding to a heartbeat phase that is close to the heartbeat phase designated in advance, as the corresponding frame.

14. The apparatus according to claim 1, wherein the selector specifies the corresponding frame from among the group of frames, based on raw data used for reconstructing images corresponding to the plurality of heartbeat phases.

15. The apparatus according to claim 1, wherein, when having received from an operator a change instruction indicating that the specified corresponding frame should be changed, the selector learns a heartbeat phase of a corresponding frame after the change.

16. An X-ray Computed Tomography (CT) apparatus comprising:
a generating unit that generates a group of frames corresponding to images that correspond to a plurality of heartbeat phases and that are reconstructed from acquired image data that is acquired during one heart beat;
a selector that specifies a corresponding frame that corresponds to a specific heartbeat phase from among the group of frames;
a first boundary detecting unit that detects a boundary of the heart from the corresponding frame; and
a second boundary detecting unit that detects a boundary of the heart from each of the frames other than the corresponding frame, by using the detected boundary in the corresponding frame.

17. The X-ray CT apparatus according to claim 16, wherein the generating unit generates the group of frames corresponding to the images that correspond to the plurality of heartbeat phases, by reconstructing the images that correspond to the plurality of heartbeat phases from acquired image data that is acquired after a second characteristic wave following a first characteristic wave that serves as a trigger for starting an X-ray radiation.

18. The apparatus according to claim 17, wherein the generating unit acquires the acquired image data during one heart beat, the acquired image data being acquired by reconstructing data corresponding to the plurality of heartbeat phases of an entirety of the heart.

19. The apparatus according to claim 17, wherein the generating unit performs a reconstructing process on a set made up of pieces of acquired image data each of which is centered on a reconstruction center phase and each of which is extracted from the acquired image data with respect to a different one of the plurality of heartbeat phases.

20. An image processing apparatus comprising:
a circuitry that generates a group of frames corresponding to reconstructed images that correspond to a plurality of heartbeat phases of a heart of a subject;
a circuitry that specifies a corresponding frame that corresponds to a specific heartbeat phase from among the group of frames;
a circuitry that detects a boundary of the heart from the corresponding frame; and
a circuitry that detects a boundary of the heart from each of the frames other than the corresponding frame, by using the detected boundary in the corresponding frame.
